(19) United States
(12) Patent Application Publication
(10) Pub. No.: US 2026/0207676 A1
(43) Pub. Date: Jul. 23, 2026

(54) LUNG CELL TRANSPLANTATION FOR THE TREATMENT OF LUNG FIBROSIS

(71) Applicant: Board of Regents, The University of Texas System, Austin, TX (US)

(72) Inventors: Yair Reisner, Austin, TX (US); Irit Milman Krentsis, Austin, TX (US); Yangxi Zheng, Austin, TX (US)

(21) Appl. No.: 19/143,278

(22) PCT Filed: Jan. 24, 2024
(86) PCT No.: PCT/US2024/012803

§ 371 Date: Jun. 25, 2025

Related U.S. Application Data
(60) Provisional application No. 63/441,122, filed on Jan. 25, 2023.

(51) Int. Cl. A61K 35/42; A61K 45/06; A61P 11/00; C12N 5/00; C12N 5/071; G01N 33/68

(52) U.S. Cl. CPC A61K 35/42; A61K 45/06; A61P 11/00; C12N 5/0087; C12N 5/0689; G01N 33/6872; G01N 2333/70596; G01N 2800/52

(57) ABSTRACT

Provided herein are lung forming progenitor cell therapies which do not require pre-conditioning treatments. Administration of the progenitor cells can replace or replenish populations of host-derived patch forming cells to reverse or inhibit fibrosis, promote healing, and improve lung function. Further disclosed herein are methods for monitoring fibrosis, and for determining whether a subject will be receptive to progenitor cell treatments.

### CROSS REFERENCE TO RELATED APPLICATIONS

The present application claims the benefit of priority under U.S.C. § 119(e) to U.S. Provisional Application Ser. No. 63/441,122, filed on Jan. 25, 2023, the entire contents of which is incorporated herein by reference in its entirety.

### BACKGROUND OF THE INVENTION

### Field of the Invention

The present invention relates generally to pulmonary fibrosis and more specifically to a method of treating a subject with pulmonary fibrosis by transplantation of lung forming progenitor cells.

### Background Information

Chronic pulmonary diseases, including chronic obstructive pulmonary disease (COPD) and idiopathic pulmonary fibrosis (IPF) are among the most common non-communicable diseases and the leading causes of morbidity and mortality, and has an estimated prevalence of 13 to 20 per 100,000 people worldwide. About 100,000 people are affected in the United States, and 30,000 to 40,000 new cases are diagnosed each year. IPF has a poor prognosis with a median survival of 3.8 years from time of diagnosis in adults aged 65 years or older. IPF is characterized by atypical interstitial pneumonia pattern on high-resolution CT scanning, as manifested by basal predominant reticulation, traction bronchiectasis and honeycombing. Histologically, these features are represented by heterogenous paraseptal fibrosis, architectural distortion, and fibroblastic foci. Although the antifibrotic drugs pirfenidone and nintedanib can slow disease progression, often, the only treatment with the potential to improve quality of life and survival is lung transplantation. However, the shortage of lungs suitable for transplantation and the high mortality risk associated with the procedure has led to an extensive search for lung stem cell populations which can potentially offer alternative sources for transplantation and less invasive treatment.

Various cell populations exhibit regenerative potential, including BM-derived cells, lung-derived p63+ cells, LNEP (lineage negative epithelial progenitors) and mouse and human Sox9+ cells. Recently, fetal and adult lung forming progenitor cells have been shown to potentially offer an attractive source for transplantation in mice, provided that the lung stem cell niche in the recipient is vacated of endogenous lung forming progenitor cells by adequate conditioning. Following lung injury with NA (naphthalene) or CY (cyclophosphamide), endogenous host lung progenitors, which are generally quiescent, exhibit a robust proliferative response, competing with donor progenitors and preventing engraftment. However, these endogenous proliferating cells are radio-sensitive, and can be eliminated by subsequent sublethal 6 Gy total body irradiation (TBI). Thus, in a procedure akin to bone marrow transplantation (BMT), a single cell suspension of mouse or human fetal lung cells harvested at the canalicular phase of gestation (20-22 weeks in human, and E15-E16 for mouse) and infused intravenously (I.V) following conditioning of recipient mice with naphthalene and 6 Gy TBI, led to marked lung chimerism within alveolar and bronchiolar lineages. This chimerism is associated with significantly improved lung function. In particular, transplantation of fetal or adult lung cells following such conditioning leads to extensive donor derived ‘patches’ with bronchiolar, alveolar and endothelial cell lineages. More recently, this approach for lung chimerism induction was extended to transplantation of a single cell suspension of adult mouse lung donors requiring about three-fold higher cell doses to attain a similar level of chimerism. Notably, in these studies in which donor derived cells originated from GFP+ or TdTomato+ donors, the infused lung forming progenitor cells colonized in discrete green or red patches with multiple cell lineages, including epithelial and endothelial cells.

### SUMMARY OF THE INVENTION

The present invention is based on the seminal discovery that pre-conditioning regimens are not requisite for lung forming progenitor cell therapies. In particular, certain lung pathologies can promote receptivity to donor cell engraftment, enabling progenitor cell therapies which do not require pre-conditioning through stem cell depletion. Based on graft failure in non-preconditioned mouse models, it was previously believed that pre-conditioning was required for lung engraftment therapy. However, it was surprisingly discovered herein that certain lung pathologies remove the requirement for pre-conditioning, allowing lower intensity treatment regimens for subjects with these conditions. The present disclosure provides studies in different mouse models using a single cell lung suspension which suggest that in a normal recipient, conditioning is required while in two models of lung fibrosis, conditioning is not required. The present invention provides progenitor cell therapies which do not include pre-conditioning steps, and which are therefore accessible to a broader population of lung fibrosis patients.

In one embodiment, the present invention provides a method of treating pulmonary fibrosis in a subject which includes administering to a subject having at least about 15% lung fibrosis a therapeutically effective amount of lung forming progenitor cells, thereby treating pulmonary fibrosis in the subject.

In one aspect, prior to administering, a level of fibrosis in the lung of the subject is measured by comparing a fibrosis volume in the lung to a total lung volume. In another aspect, the measuring includes subjecting the subject to a computed tomography (CT) scan and/or assessing the level of fibrosis by a lung biopsy sample. In some aspects, assessing the level of fibrosis on a lung biopsy sample includes subjecting the lung biopsy sample to an Ashcroft test. In some aspects, the treatment is administered in the absence of a pre-conditioning treatment prior to the administration of the cells. In certain aspects, the method further includes dissociating a pulmonary tissue to obtain lung forming progenitor cells. In particular aspects, dissociating the pulmonary tissue includes subjecting the pulmonary tissue to an enzymatic digestion. In certain aspects, the pulmonary tissue is a fetal pulmonary tissue or adult pulmonary tissue. In some aspects, the pulmonary tissue is a human pulmonary tissue. In some aspects, the cells are autologous cells. In some aspects, the cells are allogenic cells. In some aspects, the cells are administered in a cell suspension. In various aspects, the method further includes enriching the cells for those expressing an endothelial marker and/or an epithelial marker and/or depleting the cells of cells expressing CD45. In some aspects, the epithelial marker is selected from CD326, CD324, CD245, aquaporin-5, podoplanin, or advanced glycosylation end-product specific receptor. In certain aspects, the endothelial marker is CD31, CD144, or ERG. In various aspects, the method further includes enriching the cells for those that are CD326⁺CD31⁺, CD324⁺CD31⁺, CD326⁺CD144⁺, and/or CD324⁺CD144⁺. In certain aspects, the method further includes determining that the cells include CD326⁺CD31⁺, CD324⁺CD31⁺, CD326⁺CD144⁺, and/or CD324⁺CD144⁺ cells. In certain aspects, the method further includes depleting the cells of T cells, depleting the cells of B cells, or a combination thereof. In one aspect, enriching the cells or depleting the cells includes contacting the cells with an agent that binds to the epithelial marker, the endothelial marker, or CD45. In another aspect, the binding agent is an antibody. In some aspects, the method further includes expanding the isolated pulmonary cells in culture. In various aspects, the subject has idiopathic pulmonary fibrosis or chronic obstructive pulmonary disease. In certain aspects, the cells are administered in one or more dose.

In some aspects, the method further comprises determining expression of an epithelial marker, an endothelial marker, or a combination thereof in the cells. In some aspects, the epithelial marker is selected from CD326, CD324, CD245, aquaporin-5, podoplanin, or advanced glycosylation end-product specific receptor. In some aspects, the epithelial marker is CD326. In some aspects, the endothelial marker is CD31, CD144, or ERG. In some aspects, the endothelial marker is CD31. In some aspects, a predetermined proportion of the cells express the epithelial marker and the endothelial marker. In some aspects, the predetermined proportion is least about 0.1%, at least about 1%, at least about 2%, at least about 5%, at least about 10%, at least about 20%, at least about 30%, at least about 40%, at least about 50%, at least about 60%, at least about 70%, at least about 80%, at least about 90%, or at least about 95% of the cells. In some aspects, the epithelial marker is CD326 and the endothelial marker is CD31. In some aspects, the cells are expanded for at least about 12 hours, at least about 24 hours, at least about 36 hours, at least about 48 hours, at least about 72 hours, at least about 120 hours, at least about 180 hours, at least about 240 hours, at least about 300 hours, or at least about 600 hours prior to the determining. In some aspects, the cells are determined to not be suitable for administration to the subject when a proportion of the cells which do not express the epithelial marker and the endothelial marker exceeds a predetermined threshold. In some aspects, the predetermined threshold is at least about 0.1%, at least about 1%, at least about 2%, at least about 5%, at least about 10%, at least about 20%, at least about 30%, at least about 40%, at least about 50%, at least about 60%, at least about 70%, at least about 80%, at least about 90%, or at least about 95% of the cells.

In a further aspect, the method further includes administering the subject an immunosuppressive therapy. In particular aspects, the immunosuppressive therapy includes tacrolimus, everolimus, sirolimus, rapamycin, cyclosporine A, an anti-lymphocyte globulin antibody, an anti-thymocyte globulin (ATG) antibody, an anti-CD3 antibody, a steroid, azathioprine, an anti-IL-2Ra receptor antibody, mycophenolic acid, or an anti-CD20 antibody.

In another embodiment, the invention provides a method of improving lung function in a subject in need thereof which includes administering to a subject having at least about 15% lung fibrosis a therapeutically effective amount of lung forming progenitor cells, thereby improving lung function in the subject.

In one aspect, prior to administering, a level of fibrosis in the lung of the subject is measured by comparing a fibrosis volume in the lung to a total lung volume. In another aspect, improving lung function includes reducing fibrosis and/or preventing or slowing down fibrosis progression. In some aspects, improving lung function includes improving respiratory system Newtonian resistance, lung tissue compliance, lung tissue elastance, tissue dampening and/or forced expired volume. In certain aspects, the subject has pulmonary fibrosis or chronic obstructive pulmonary disease. In some aspects, treatment is administered in the absence of a pre-conditioning treatment prior to the administration of the cells. In some aspects, the cells are administered in a cell suspension.

In another embodiment, the invention provides a method of identifying a subject with pulmonary fibrosis as a candidate for treatment with lung forming progenitor cells which includes a) measuring the level of fibrosis in the lungs of the subject; and i) classifying a subject with a level of fibrosis of at least about 15% or greater as a likely responder to cell transplantation, thereby identifying the subject as suitable for cell transplantation, or ii) classifying a subject with a level of fibrosis of about 0 to less than 15% as a likely non-responder to lung cell transplantation, thereby identifying the subject as unsuitable for cell transplantation, thereby identifying a subject with pulmonary fibrosis as a candidate for treatment with lung forming progenitor cells.

In one aspect, measuring the level of fibrosis is by a computed tomography (CT) scan and/or assessing the level of fibrosis on a lung biopsy sample. In a further aspect, the method further includes administering a cell suspension with isolated lung forming progenitor cells to the subject identified as a likely responder. In various aspects, administering the cell suspension includes increasing a number of donor-derived patch forming cells and/or decreasing a number of host-derived patch-forming cells. In certain aspects, the donor-derived patch forming cells express both endothelial and epithelial markers. In some aspects, the endothelial marker is CD31, CD144, or ERG. In some aspects, the endothelial marker is CD31. In some aspects, the epithelial marker is CD326, CD324, CD245, aquaporin-5, podoplanin, or advanced glycosylation end-product specific receptor. In some aspects, the epithelial marker is CD326. In some aspects, the method further includes measuring a second level of fibrosis in the lungs of the subject at a second time point and (i) maintaining the subject as an unlikely responder if the second level of fibrosis is of about 0 to less than 15%; or (ii) reclassifying the subject as a likely responder if the second level of fibrosis is of at least about 15% or greater. In some aspects, a level of fibrosis of at least about 15% or greater is indicative of a reduction in a number of endogenous lung stem cells sufficient to ensure engraftment of the lung forming progenitor cells. In certain aspects, a level of fibrosis of about 1 to less than 15% is indicative of an absence of a sufficient reduction in a number of endogenous lung stem cells to allow engraftment of the lung forming progenitor cells.

### BRIEF DESCRIPTION OF THE DRAWINGS

FIGS. 1A-1C are a schematic illustration, set of images, and plot that illustrate number of patch forming lung forming progenitor cells, remaining in mice treated with BLM for different periods. This is tested by transplantation of different number of lung cells from BLM treated mice into pre-conditioned recipient mice FIG. 1A is a schematic representation of the study design. FIG. 1B is a graph illustrating the number of TdTomato and GFP positive donor derived patches after transplantation of different numbers of lung cells from mice treated with BLM for different periods (size scale=500 um). FIG. 1C is a plot that shows quantitative analysis of the number of donor derived patches per 2 mm² lung area after transplantation of different numbers of lung cells from mice treated with BLM for different periods.

FIGS. 2A-2D are a graphic illustration, images, and a plot that show donor derived patches after transplantation of TdTomato+lung cells into mice treated with BLM for different periods. FIG. 2A is a schematic representation of the experimental plan. FIG. 2B is a graph illustrating the number of donor derived lung patches per 2 mm² lung area in mice treated with BLM for different intervals. FIG. 2C is a set of images illustrating typical example of donor derived lung patches in large lung area of mice treated with BLM for different intervals. FIG. 2D is a plot that shows percentage of chimeric mice, defined by at least 4 visible donor derived patches consisting of more than 20 donor cells each.

FIGS. 3A-3E are a set of images and plots overviewing typical cell compositions of GFP+ donor derived patches in the lungs of transplanted mice pre-treated with BLM for 4 weeks. FIG. 3A is a set of images of typical immunohistological stains of HOPX+ AT1 alveolar cells. FIG. 3B is a set of images of typical immunohistological stains of LAMP3+ AT2 alveolar cells. FIG. 3C is a set of images of typical immunohistological stains of ERG+ endothelial cells. FIG. 3D is a set of images of typical immunohistological stains of PDGRa+ mesenchymal cells. FIG. 3E is a series plots of average percentages of different cell types in donor derived lung patches, representing a minimum of 40 patches for each staining obtained from 3-5 chimeric mice.

FIGS. 4A-4H are a schematic illustration and a set of images and plots that illustrate fibrosis attenuation and functional benefit in the BLM mouse model at 8 weeks after transplantation of lung cells. FIG. 4A is a schematic representation of the experimental plan. FIG. 4B is a photograph illustrating typical IHC staining of lung tissue of mice preconditioned for 4 weeks with BLM and transplanted one week later with TdTomato+ and GFP+ C57BL donor derived lung cells. Lung tissue was harvested at 8 weeks post transplant (scale bar=200 um). FIG. 4C shows photographs illustrating typical three-chrome staining of lung treated with vehicle (PBS, left), bleomycin (middle) or bleomycin with transplantation with total 8*10⁶ donor derived cells (right). All groups were tested for fibrosis at the same time, namely, at 13 weeks after initiation of BLM treatment. FIG. 4D is a graph illustrating Ashcroft test comparing fibrosis levels in different groups of mice based on Mason-Tri-chrome staining. FIG. 4E is a graph illustrating lung tissue volume percent measured by CT. FIG. 4F shows graphs illustrating functional parameters as measured by FlexiVent (one-way Anova with Dunnett test was used for statistical analysis; *p<0.03, **p<0.002, ***p<0.0002, ****p<0.0001). FIG. 4G is a plot of chimerism level defined by average patch number per 2 mm² lung area. Mice exhibiting less than an average of four patches per 2 mm² of lung tissue (purple) were excluded from further comparisons. FIG. 4H is a plot of total collagen in the paraffin-embedded lung tissue by hydroxyproline assay, from one transplantation experiment.

FIGS. 5A-5H is a set of illustrations, images and plots that show donor derived patches after transplantation of TdTomato+ lung cells into TRF1ko/SPC mice treated with TMX after different time periods. FIG. 5A is a schematic representation of the experimental plan showing induction of TRF1 knock-out in AT2 cells by TMX treatment 3 times a week for 6.5 weeks. Thereafter mice were transplanted with lung cells from TdTomato+ donors at different time points of fibrosis progression. FIG. 5B is a graph illustrating the number of donor derived lung patches per 2 nm² lung area in mice treated with donor lung cells at different time points after TMX induction. FIG. 5C shows photographs illustrating typical example of donor derived lung patches in large lung area of mice treated with donor lung cells at different time points after TMX induction. FIG. 5D is a scheme showing generation of the SPC-Cre TRF1^{fl/fl} mouse in which TMX induced Cre recombination leads to deletion of TRF1 specifically in SPC+ AT2 alveolar cells. FIG. 5E is a plot of numbers of donor-derived lung patches per 2 mm² lung area in mice treated with donor lung cells at different time points after TMX induction. Each dot represents the average number of patches per 2 mm² based on at least 10 measurements in individual mice. FIG. 5F is a plot of percent fibrosis as measured by micro CT at different time points after initiation of TMX treatment in the absence of lung cell transplantation. FIG. 5G is a plot that shows the percentage of mice exhibiting donor chimerism as a function of time. FIG. 5H is set of representative 3D lung images generated by micro CT imaging of non-treated lung tissue (upper) and lung tissue 14 weeks after initiation of TMX administration (lower). And with tissue volume representing the fibrotic area shown with contrast and calculated for every image.

FIGS. 6A-6E are a set of plots and images that show typical cell compositions of a GPF+ donor derived patches in the lung of TRF1ko/SPC mice model transplanted 14 week after TMX induction of TRF1 knockout in AT2 cells. FIG. 6A is a set of images of typical immunohistological stains of HOPX+ AT1 alveolar cells at low magnification (left, 10 μm scalebar), zoomed in view of HOPX and GFP stains (second from left, 2 μm scalebar), HOPX and nucleus stains (second from right, 2 μm scalebar), and double staining for GFP and nuclei (right, 2 μm scalebar). FIG. 6B is a set of images of typical immunohistological stains of LAMP3+ AT2 alveolar cells at low magnification (left, 10 μm scalebar), zoomed in view of LAMP3 and GFP stains (second from left, 2 μm scalebar), LAMP3 and nucleus stains (second from right, 2 μm scalebar), and double staining for GFP and nuclei (right, 2 μm scalebar). FIG. 6C is a set of images of typical immunohistological stains of ERG+ endothelial cells at low magnification (left, 10 μm scalebar), zoomed in view of ERG and GFP stains (second from left, 2 μm scalebar), ERG and nucleus stains (second from right, 2 μm scalebar), and double staining for GFP and nuclei (right, 2 μm scalebar). FIG. 6D is a set of images of typical immunohistological stains of PDRGa+ mesenchymal cells at low magnification (left, 10 μm scalebar), zoomed in view of PDRGa and GFP stains (second from left, 2 μm scalebar), PDRGa and nucleus stains (second from right, 2 μm scalebar), and double staining for GFP and nuclei (right, 2 μm scalebar). FIG. 6E is a set of plots that show prevalences of multiple cell types in donor-derived lung patches.

FIGS. 7A-7I are a schematic and set of plots and images that show fibrosis attenuation and functional benefit in the TRF1ko/SPC mice transplanted 14 weeks after beginning of TMX induction of TRF1 knockout in AT2 cells. FIG. 7A shows an experimental plan with induction of TRF1 knock-out in TRF-/-SPEC-Cre mice and transplantation 7 weeks after the TMX induction with mixture of GFP and TdTomato fluorescent C57BL mice. FIG. 7B is a photograph illustrating typical fibrosis determined by Trichrome staining 14 weeks after beginning of TMX induction. FIG. 7C is a graph illustrating Ashcroft test showing statistically significant lower level of fibrosis in transplanted compared to non-transplanted mice (P<0.002). FIG. 7D shows graphs illustrating improved lung function in transplanted compared to non-transplanted mice measured using Flexivent to measure for Resistance (left, P<0.03), Tissue Damping (second from left, P<0.002), Tissue Elastance (second from right, P<0.03), and Forced Expired Volume (right). FIG. 7E is a set of images of typical examples of fibrosis determined by Trichrome staining 22 weeks after initiation of TMX induction (showing higher and low magnification, scale bar=200 μm and 100 μm, respectively). FIG. 7F is a plot of total collagen in paraffin embedded lung tissue, measured by the Hydroxyproline assay. FIG. 7G is an image of fibrosis determined by Trichrome staining 22 weeks after initiation of TMX induction. FIG. 7H is a plot of chimerism level defined by average patch number per 2 mm² lung area. FIG. 7I is a fluorescence image of transplanted lung tissue 8 weeks after transplantation of a 1:1 mixture of TdTomato+ and GFP+ C57BL donor-derived lung cells, following 14 weeks of host pre-conditioning. Scale bar=200 μm.

FIG. 8 is a graphic illustration that shows a conditioning scheme for lung injury in C57BL/6 mice.

FIGS. 9A-9D show embryonic precursor lung cell engraftment following different lung pre-conditioning regimens. FIG. 9A shows a fluorescence microscopy image of lungs from C57BL/6 adult mice pre-treated with 6 Gy TBI and transplanted with GFP+ embryonic precursor lung cells. Lungs were harvested at 8 weeks post-transplant. FIG. 9B shows a fluorescence microscopy image of lungs from C57BL/6 adult mice pre-treated with Naphthalene and transplanted with GFP+ embryonic precursor lung cells. Lungs were harvested at 8 weeks post-transplant. FIG. 9C shows a fluorescence microscopy image of lungs from C57BL/6 adult mice pre-treated with Naphthalene and 6 Gy TBI and transplanted with GFP+ embryonic precursor lung cells. Lungs were harvested at 8 weeks post-transplant. FIG. 9D shows results from quantitative morphometric analysis of GFP+ patches of engrafted cells per mm³ lung tissue after 6 Gy radiotherapy, naphthalene, or 6 Gy radiotherapy plus naphthalene conditioning regimens.

FIGS. 10A-10I are plots and images that show assessment of durable BLM-induced lung fibrosis. FIG. 10A is a set of typical micro CT 3D images of lung tissue from mice following treatment with PBS (left) and at 8 weeks after completion of BLM treatment (right). FIG. 10B is a plot of percent fibrotic tissue calculated based on micro CT scans at 8 weeks after completion of BLM treatment. FIG. 10C is a plot showing quantitative measurements of total collagen in paraffin embedded lung tissue as measured by hydroxy-proline assay. FIG. 10D is a set of images of representative H&E lung stains at 8 weeks after completion of BLM treatment (middle and bottom row) as compared to treatment with PBS (upper row). FIG. 10E is a set of images of representative trichrome lung stains at 8 weeks after completion of BLM treatment (middle and bottom row) as compared to treatment with PBS (upper row). FIG. 10F is a set of images of representative Alpha Sma lung stains at 8 weeks after completion of BLM treatment (middle and bottom row) as compared to treatment with PBS (upper row). FIG. 10G is a set of images of representative fibronectin lung stains at 8 weeks after completion of BLM treatment (middle and bottom row) as compared to treatment with PBS (upper row). FIG. 10H is a set of images of representative collagen IV lung stains at 8 weeks after completion of BLM treatment (middle and bottom row) as compared to treatment with PBS (upper row). FIG. 10I is a set of plots of lung function assessments (from left to right, Elastance (E), Resistance (R), Tissue Damping (G), Tissue Elastance (H), and Forced Expired Volume (FEV_{0.1}) measured by Flexivent at different time points after completion of BLM treatments.

FIGS. 11A-11C are images of typical tri-chrome and alpha-SMA staining of mouse lungs 8 weeks after completion of BLM treatment. FIGS. 11A-B are 200 μm (left) and 500 μm (middle and right) images of tri-chrome-stained lung tissue from BLM-treated mice. FIG. 11C is a 200 μm (left) and a set of 500 μm (middle and right) images of alpha-SMA-stained lung tissue from BLM-treated mice.

FIG. 12 is a set of images of typical examples of donor-derived lung patches in whole lung of mice treated with BLM for 4 weeks and transplanted with 8 million TdTomato+ lung cells. Each panoramic view represents a different transplanted mouse (Scale size=500 μm).

FIGS. 13A-13D are images that track single cells the Z axis within donor derived patches found in the lung of BLM treated mice at 8 weeks after transplantation of GFP+ lung cells. Chimeric lung tissue was stained for expression of GFP and different markers and analyzed by confocal microscopy through optical slice z=1 to z=10. Nuclei were traced by Hoechst staining in GFP+ donor derived cells. AT1 and AT2 alveolar cells, endothelial and mesenchymal cells are delineated by staining for HOPX, LAMP, ERG and PDGRa respectively. Left column showing low magnification of donor derived patch (scale bar=10 μm) and on the right it shown the higher magnification double positive cells (scale bar=3 μm). FIG. 13A is a set of images with HOPX staining. FIG. 13B is a set of images with LAMP3 staining. FIG. 13C is a set of images with ERG staining. FIG. 13D is a set of images with PDGRa staining.

FIG. 14 is a set of images of panoramic views of donor derived lung patches in SPC-Cre TRF1^{fl/fl} recipients of TdTomato+ lung cells. Mice were treated with TMX for 7 weeks and transplanted with 8 million lung cells from TdTomato+ donors. Chimeric lungs were harvested 8 weeks after transplantation. Scale bar 200 μm, N=8 mice.

FIGS. 15A-15D are images that track single cells across the Z axis within donor derived patches found in SPC-Cre TRF1fl/fl recipients of TdTomato+ lung cells. Chimeric lung tissue was stained for expression of GFP and different markers and analyzed by confocal microscopy through optical slice z=1 to z=6. Nuclei were traced by Hoechst staining in GFP+donor derived cells. AT1 and AT2 alveolar cells, endothelial and mesnchymal cells are delinated by staining for HOPX, LAMP3, ERG and PDGRa, respectively. FIG. 15A is a set of images with HOPX staining. FIG. 15B is a set of images with LAMP3 staining. FIG. 15C is a set of images with ERG staining. FIG. 15D is a set of images with PDGRa staining.

FIGS. 16A-16B are images of hyaluronic acid stains of lungs before and after transplantation. FIG. 16A is a set of images of chimeric lungs harvested 8 weeks after transplantation and stained for HA. The upper row shows typical staining at 500 μm magnification of mice not induced with 4 weeks of BLM (PBS), mice induced with BLM and mice induced with BLM, and transplanted. The lower row shows 200 μm magnification of the boxed-in areas indicated in the top row. FIG. 16B is a set of images of HA staining of mice treated with TMX for 7 weeks and transplanted with 8 million lung cells from TdTomato+ donors post another 7 weeks. The upper row shows typical staining at 500 μm magnification of non TMX (PBS), mice induced with TMX, mice treated with TMX, and transplanted. The lower row shows 200 μm magnification of the boxed-in areas indicated in the top row.

FIGS. 17A-17C are sets of images that show incorporation of donor derived AT1, AT2 and endothelial cells into alvoli of BLM treated mice at 2 months after transplantation of lung single cell suspension from GFP+ donors. FIG. 17A is a set of images of Hoechst and HOPX stains. FIG. 17B is a set of images of Hoechst and LAMP3 stains. FIG. 17C is a set of images of Hoechst and ERG stains. The leftmost image in each of FIGS. 17A-C include GFP stains to highlight donor cells. The second image from the left in each of FIGS. 17A-C include nuclear stains. The second image from the right in each of FIGS. 17A-C include a stain for a tested lineage marker. The rightmost image in each of FIGS. 17A-C shows colocalization of donor-derived cells.

### DETAILED DESCRIPTION OF THE INVENTION

The present invention is based on the seminal discovery that pre-conditioning regimens are not requisite for lung forming progenitor cell therapies in subjects with certain lung pathologies.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “and/or” includes any and all combinations of one or more of the associated listed items.

As used herein, the term “about” in association with a numerical value is meant to include any additional numerical value reasonably close to the numerical value indicated. For example, and based on the context, the value can vary up or down by 5-10%. For example, for a value of about 100, means 90 to 110 (or any value between 90 and 110).

As used herein and in the claims, the terms “comprising,” “containing,” and “including” are inclusive, open-ended and do not exclude additional unrecited elements, compositional components or method steps. Accordingly, the terms “comprising” and “including” encompass the comparably more restrictive terms “consisting of” and “consisting essentially of.”

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

The present invention provides cell transplant therapies for the treatment of pulmonary fibrosis. It was previously believed that pre-conditioning regimens were essential to achieve lung forming progenitor cell engraftment. However, it was surprisingly discovered herein that moderate and advanced stages of fibrosis can improve receptivity to donor cell engraftment, enabling regenerative progenitor cell transplantation without pre-conditioning. As was further shown herein (for example in EXAMPLE 3), these treatments not only replenish multiple stem cell phenotypes, but can also improve overall lung health and mitigate fibrosis. Accordingly, the therapies disclosed herein provide an improved treatment option that does not require damage induction through pre-conditioning to foster cell engraftment and fibrosis repair.

As used herein, the terms “fibrosis” and “fibrotic” refer to fibrous tissue levels beyond those observed in healthy tissues. For example, fibrosis can denote extracellular matrix (ECM) and collagen deposits at levels which diminish elasticity and inhibit normal tissue function. Fibrosis can include fibroblast overaccumulation or overactivity in an organ or tissue. Fibrosis represents By treating fibrosis, it is meant that a degree of fibrosis is diminished (e.g., a decrease in collagen and/or ECM levels or diminished Ashcroft score) or that a rate of fibrosis is retarded in a subject, organ, or tissue.

As used herein, the term “pulmonary fibrosis” refers to fibrosis of a respiratory tract. Examples of pulmonary fibrosis and diseases which can include pulmonary fibrosis, but are not limited to, cystic fibrosis, emphysema, chronic obstructive pulmonary disease (COPD), idiopathic pulmonary fibrosis, lung cancer, acute lung injury, respiratory distress syndrome, chronic lung disease, and chronic lung inflammation.

As used herein, the term “fibroblast” can denote a connective tissue cell active in extracellular matrices (ECMs) and collagen deposition.

As used herein, the term “pulmonary tissue” refers to tissue of the respiratory tract. In particular cases, pulmonary tissue refers to tissue of the lower respiratory tract, including tissue of the lungs, trachea, bronchi, bronchioles, and alveoli. Pulmonary tissue can be tissue which is in or obtained from the respiratory tract, or which is generated from respiratory cells.

The present invention is based on the seminal discovery that pre-conditioning regimens are not requisite for lung forming progenitor cell therapies, allowing these therapies to be performed with pre-conditioning, thereby diminishing the intensiveness of such therapies and expanding the subset of IPF subjects who are candidates for this treatment. At present, the primary options available for IPF treatment are relatively high-risk lung transplantations. Despite the emergence of novel compounds for the treatment of IPF patients, lung transplantation remains the only “curative” option for IPF. The low 5-year mean survival rate following transplantation highlights the urgent need for novel therapies.

The presently disclosed studies, which investigated the efficacy of lung stem cell transplantation in two distinct models of lung fibrosis, show that lung cell transplantation is efficacious histologically, biochemically, radiographically, and physiologically. These involved 3 independent transplantation experiments in each model, including at least 2 for functional analysis. It was found in most transplanted mice that donor-derived patches occupy a substantial volume of the lungs, with more than 12 donor-derived patches per 2 mm² of lung area, following transplantation of a lung cell suspension into mice exhibiting moderate levels of lung fibrosis. Immunohistochemical analysis showed that in both fibrosis models, that the numerous donor-derived patches include AT1 (HOPX+) and AT2 (LAMP-3+, SPC+) alveolar cells, as well as endothelial (ERG⁺) and mesenchymal (PDGRα⁺) cells. Furthermore, functional analysis measuring lung tissue resistance, elastance and forced expired volume showed that this robust chimerism is associated with improved lung function in all parameters. Pathological analysis of the tissue also revealed a notable reduction in fibrotic tissue compared to non-treated mice.

Notably, in both models, the progression of lung fibrosis was associated with marked depletion of endogenous patch-forming lung progenitors in the recipients, demonstrating receptivity to donor cell engraftment without need for further conditioning. This surprising finding supports a model akin to bone marrow transplantation (BMT) in which stem cell competition for lung stem cell niches represents a major barrier for attaining lung chimerism following lung stem cell transplantation. The lack of stem cell competition during fibrosis progression in two mouse models is reminiscent of BMT in patients with severe combined immunodeficiency (SCID). These patients bear genetically defective T cell progenitors that cannot compete with donor-derived normal progenitors, and which can develop in the recipient's thymus following transplantation, without any need for host stem cell ablation.

More broadly, it was determined herein that lung forming progenitor cells can populate, proliferate within, and repair fibrotic lungs, enabling a range of therapies for treating fibrosis. As fibrosis often coincides with proinflammatory immune polarization and inhibited tissue repair processes, many fibrotic subjects are unable to receive preconditioning treatments required by traditional cell transplant procedures, and thus have lacked options for managing and treating their fibrosis. The compositions and methods disclosed herein provide treatment and management options for previously untreatable forms of fibrosis. Furthermore, as many subjects with lung pathologies have diminished capacities to recover from pre-conditioning regimens, the methods disclosed herein can increase the likelihood of positive treatment outcomes.

The presently disclosed studies demonstrate that a single i.v. infusion of a lung cell suspension can lead to robust regeneration of both donor-derived AT2 and AT1 alveolar cells, as well as endothelial cells. Considering that all major fibrotic diseases of the lung involve not only epithelial injuries but are also associated with vascular and endothelial damage (leading to poor oxygen exchange in the lungs), this multi-lineage engraftment could be particularly valuable and offers advantages over AT2 cell transplantation for lung function repair.

Leveraging these discoveries, in one embodiment, the present invention provides a method of treating pulmonary fibrosis in a subject which includes administering to a subject having at least about 15% lung fibrosis a therapeutically effective amount of lung forming progenitor cells, thereby treating pulmonary fibrosis in the subject.

As used herein, the term “subject” refers to any individual or patient to which the disclosed methods are performed, to whom the disclosed compositions are administered, or from whom a biological material (e.g., a tissue sample, a cell, or a biofluid) is obtained. Generally, the subject is human, although as will be appreciated by those in the art, the subject may be a non-human animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The term “treatment” is used interchangeably herein with the term “therapeutic method” or “therapy” and refers to 1) therapeutic treatments or measures that cure, slow down, lessen symptoms of, and/or halt progression of a diagnosed pathologic conditions or disorder (e.g., idiopathic pulmonary fibrosis), and/or 2) prophylactic/preventative measures. Those in need of treatment may include individuals already having a particular medical disorder as well as those who may ultimately acquire the disorder (i.e., those needing preventive measures).

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration.

In some aspects, administering includes intravenous injection, intrapulmonary administration, intratracheal administration, intrabronchial administration, intranasal administration, nebulization, powder inhalation, intrapulmonary injection, intraperitoneal, intrathecal, or pulmonary artery infusion. In some cases, the administering includes intravenous or intrapulmonary administration.

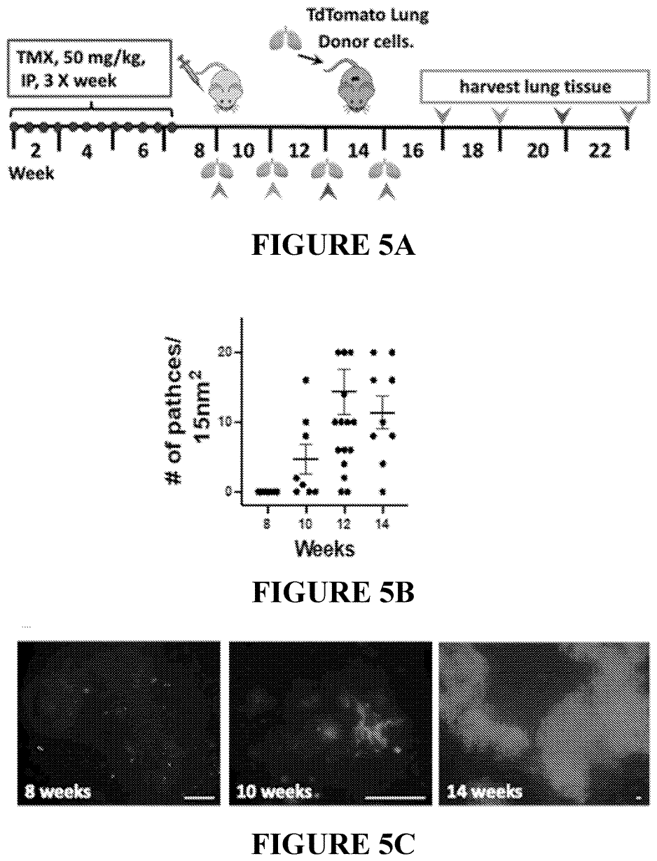

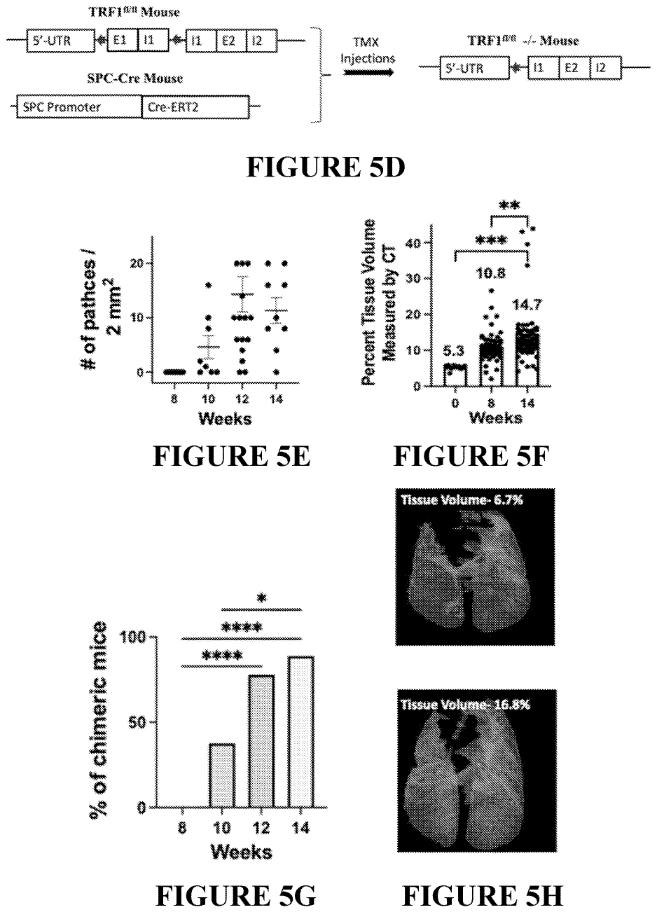

It was determined herein that fibrosis can increase donor cell engraftment receptivity, such that lung forming progenitor cell administration can lead to chimerism conducive to lung repair and fibrosis inhibition. In particular, in the BLM model, the optimal time for engraftment was found at 3-4 weeks after initiation of BLM treatment (total of 6-8 administrations of BLM), while in the SPC-Cre TRF1^{fl/fl} model, the optimal time for treatment was determined to be 6-7 weeks following completion of TMX treatment (total of 12-14 weeks from TMX treatment initiation). In contrast, only poor lung chimerism was observed in either model at earlier time points. Moreover, using CT analysis, it was shown that a level of fibrosis occupying about 15% or more of the lung is conducive to effective engraftment and colonization of the lung by donor-derived patch-forming lung progenitors (e.g., as shown in FIGS. 5E-5G). This level of fibrosis is similar to that found by CT in IPF patients with moderate fibrosis. Such patients, for whom the disease is otherwise on an invariably fatal trajectory, are likely to benefit from initiation of cellular therapy.

Building from these discoveries, in some cases, the subject has at least about 15%, at least about 16%, at least about 17%, at least about 18%, at least about 19%, at least about 20%, at least about 21%, at least about 22%, at least about 23%, at least about 24%, or at least about 25% lung fibrosis. In some cases, the subject has subject has lung honeycombing, ground-glass opacities, reticular opacities, basal predominant reticulation, traction bronchiectasis, or a combination thereof. In some cases, the lung health of the subject is prohibitive to lung pre-conditioning (e.g., by damage induction through chemical treatment or radiation).

In one aspect, prior to administering, a level of fibrosis in the lung of the subject is measured by comparing a fibrosis volume in the lung to a total lung volume. As used herein, the term “fibrosis volume” can denote an amount of space within a lung occupied by fibrotic tissue. When taken as a ratio to total lung volume, fibrosis volume can indicate the extent of fibrosis within a lung. Accordingly, fibrosis volume can be a useful for determining the severity of fibrosis in subject. In some cases, fibrosis volume denotes the volume of a lung with honeycombing, ground glass, or reticular opacities (e.g., as detailed in Bartholmai et al. J Thorac Imaging. 2013; 28:298-307.). In some cases, fibrosis volume denotes a volume of fibrotic lung parenchyma (e.g., as detailed in Radiat Oncol J. 2014; 32(1): 43-47.).

Fibrosis volume and total lung volume can be determined by numerous techniques well known in the art. In many aspects, the measuring can include subjecting the subject to a computed tomography (CT) scan and/or assessing the level of fibrosis in a lung biopsy sample. As used herein, computed tomography denotes imaging techniques which utilize X-rays and computational modeling to generate 2-dimensional cross-sectional images or 3-dimensional images of a solid structure such as a lung. Owing to its resolution and non-invasiveness, CT is commonly used for monitoring lung pathologies, including fibrosis (e.g., see Nemoto et al. Respiratory Research 2020; 21:275.). In many lung imaging applications, CT generates images with millimeter to sub-millimeter spatial resolution, for example between about 0.1 and 5 mm³, enabling nuanced differentiation of fibrotic and non-fibrotic tissues.

In some aspects, assessing the level of fibrosis on a lung biopsy sample includes subjecting the lung biopsy sample to an Ashcroft test. Used synonymously herein with “Ashcroft scoring”, the term “Ashcroft test” refers to methods which aggregate fibrosis scores from separate microscopy images to determine the extent of fibrosis in a lung. In one implementation, the Ashcroft test can score tissue from 0 to 8, with 0 indicating normal lung; 1 indicating minimal fibrous thickening of alveolar or bronchiolar walls; 3 indicating moderate thickening of walls without obvious damage to lung architecture; 5 indicating increased fibrosis with definitive damage to lung structure and formation of fibrous bands or small fibrous masses; 7 indicating severe distortion of structure and large fibrous area; and 8 indicating total fibrous obliteration of lung fields (e.g., see Ashcroft et al. J Clin Pathol. 1988; 41(4): 467-470.). Ashcroft tests typically utilize optical microscopy, such as confocal, bright field, dark field, or oblique illumination microscopy.

In some aspects, the treatment is administered in the absence of a pre-conditioning treatment prior to the administration of the cells. Donor cell engraftment and colonization are typically hindered by endogenous stem cell populations. Depleting stem cell populations through preconditioning can provide a timeframe in which donor cells can engraft and induce chimerism. Preconditioning regimens often achieve this depletion with cytotoxic conditions or agents which collaterally damage other lung cell types, requiring subsequent repair and recovery. For example, preconditioning can include the use of a cytotoxic agent such as naphthalene, irradiation, ischemia, or a combination thereof. Fibrotic lungs, which often exhibit diminished repair and immune functions, can be incapable of recovering from such pretreatment, and can therefore be unamenable to traditional cell therapies. The compositions and methods of the present invention can thus provide treatment for otherwise untreatable conditions.

As used herein, “pre-conditioning” can denote a treatment which diminishes endogenous stem cell density in a target tissue. Examples of pre-conditioning regimens include total body irradiation (TBI), partial body irradiation, chemotherapy, and chemotoxic treatment (e.g., naphthalene treatment). In many cases, pre-conditioning regimens can be distinguished from immune cytoreduction and immunosuppressive therapies, which primarily target immune cells, and may optionally be used in conjunction with the methods disclosed herein to limit graft rejection.

Accordingly, in some aspects, the treatment further includes a treatment which inhibits graft rejection. In some aspects, the treatment which inhibits graft rejection includes immune cytoreduction, immunosuppression, or a combination thereof. Graft rejection inhibitory treatment can optionally be provided before, concurrently with, and/or after cell transplantation to enhance chimerism and graft stability.

In some aspects, the subject is administered an immunosuppressive therapy. The immunosuppressive therapy can be chronic (e.g., analogous to immune suppression regiments provided to organ transplant patients) or can directly coincide or precede progenitor cell administration. As non-limiting examples, the immunosuppressive therapy can include administration of an immunomodulator such as tacrolimus, everolimus, sirolimus, rapamycin, cyclosporine A, an anti-lymphocyte globulin antibody, an anti-thymocyte globulin (ATG) antibody, azathioprine, an anti-IL-2Ra receptor antibody, mycophenolic acid, or an anti-CD20 antibody.

The progenitor cells can be derived from pulmonary tissue. Fetal and adult pulmonary tissue often contain complex cellular milieus which can include hematopoietic, endothelial, epithelial and mesenchymal progenitor cells. Following administration, these cells can be capable of localizing to pulmonary tissue, expanding, and differentiating into functional lung tissue. Often, the progenitor cells include multiple cell lines (e.g., epithelial and endothelial). However, in certain aspects, the progenitor cells are depleted of or enriched for a specific cell type.

The lung forming progenitor cells can be obtained from a range of sources. While the major source for allogenic lung cell transplantation is currently limited to cadaveric lungs, it is contemplated herein that patch-forming lung progenitors can be cultured to enable harvesting of sufficient cells from lung biopsies of from suitable donors (e.g., family members). Furthermore, as disclosed herein, induction of lung chimerism can be attained in mis-matched recipients without chronic immune suppression, for example by combining lung cell transplantation with bone marrow transplantation from the same donor. However, considering that collection of sufficient numbers of hematopoietic stem cells from cadaveric lungs can be difficult, this approach might be more feasible using live donors for the collection of ex vivo expanded lung progenitors, in conjunction with freshly isolated hematopoietic stem cells.

In certain aspects, the method includes dissociating a pulmonary tissue to obtain lung forming progenitor cells. The dissociating can liberate lung forming progenitor cells from the pulmonary tissue, which can increase its suitability for delivery and engraftment. The dissociating can include a variety of mechanical, chemical, and enzymatic means. Prior to the dissociating, the pulmonary tissue can optionally be dissected or minced with a razor. Following dissociation, reagents and cellular debris can be separated from the lung forming progenitor cells, for example with filtration, chromatography, or affinity purification.

In some aspects, dissociating the pulmonary tissue includes subjecting the pulmonary tissue to an enzymatic digestion. The enzymatic digestion can be performed with a proteolytic enzyme such as trypsin or dispase, a collagenolytic enzyme such as collagenase, an ECM-targeted enzyme such as a matrix metalloproteinase, or a combination thereof. Following enzymatic digestion, enzymes can be inactivated or separated from the cells, for example chromatographically or by filtration.

In some aspects, dissociating the pulmonary tissue includes mechanical dissociation. In many such cases, the mechanical dissociation can break the tissue into fragments of a predetermined size. The mechanical dissociation can include cutting, crushing, scraping, or extrusion through a fine filter. In some cases, the mechanical dissociation is performed prior to enzymatic degradation.

In certain aspects, the pulmonary tissue is a fetal pulmonary tissue or adult pulmonary tissue. In many aspects, the fetal pulmonary tissue or adult pulmonary tissue is derived from a lower respiratory tract, such as a portion of a lung. In some aspects, the pulmonary tissue is a human pulmonary tissue.

The tissue can be autologous (i.e., derived from the subject to which they are administered) or allogenic (i.e., derived from and delivered to separate subjects). In some aspects, the cells are autologous cells. In some aspects, the cells are allogenic cells. In some aspects, the cells are administered in a cell suspension.

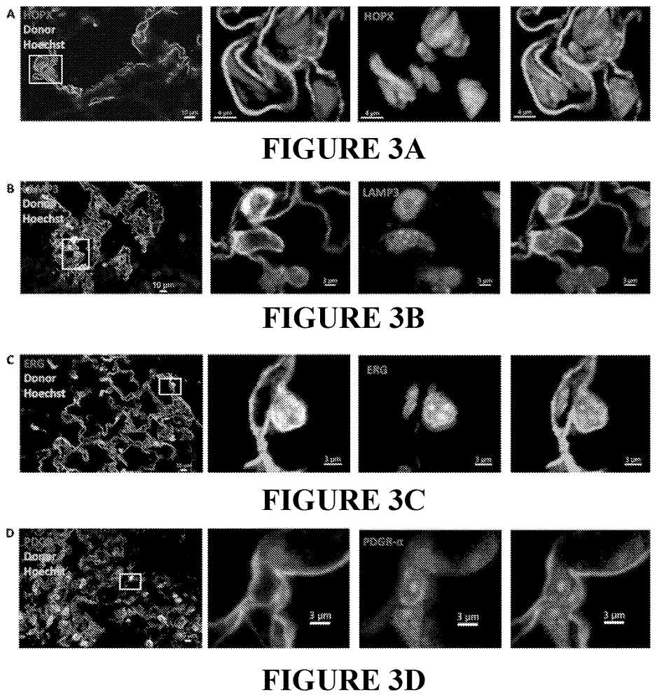

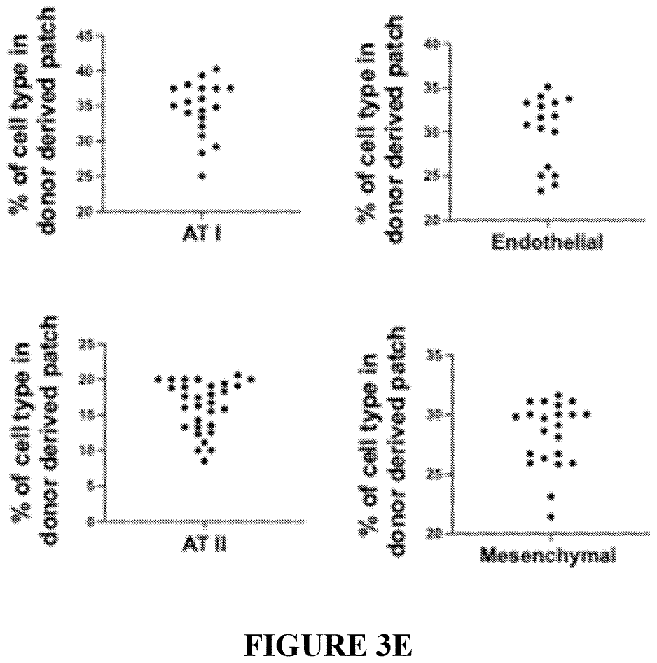

Often, the cells include multiple cell types (e.g., a combination of epithelial, endothelial, and mesenchymal progenitor cells) active in reestablishing homeostasis and inhibiting or reversing fibrosis. Furthermore, as demonstrated herein, coadministration of terminally differentiated pulmonary cells with progenitor cells often does not impact progenitor cell engraftment or antifibrotic activity. As shown in FIGS. 3A-3E, successful donor derived patches can include AT1 alveolar cells (e.g., as identified by HOPX staining or flow cytometry), AT2 alveolar cells (e.g., as identified by LAMP3 staining or flow cytometry), endothelial cells (e.g., as identified by ERG staining or flow cytometry), or mesenchymal cells (e.g., as identified by PDGRa staining or flow cytometry). It is contemplated herein that coadministration of one or more of these cells with the lung forming progenitor cells can promote successful engraftment and IPF recovery.

For example, the lung forming progenitor cells can be coadministered with between about 1% and 5% AT1 cells (i.e., AT1 cells make up a total of 1% to 5% of all cells administered to the subject). More generally, the lung forming progenitor cells can be coadministered with between about 1% and 10%, between about 1% and 15%, between about 1% and 20%, between about 1% and 25%, between about 1% and 30%, between about 1% and 40%, between about 1% and 50%, between about 1% and 60%, between about 1% and 80%, between about 5% and 10%, between about 5% and 15%, between about 5% and 20%, between about 5% and 25%, between about 5% and 30%, between about 5% and 40%, between about 5% and 50%, between about 5% and 60%, between about 5% and 80%, between 10% and 20%, between about 10% and 25%, between about 10% and 30%, between about 10% and 40%, between about 10% and 50%, between about 10% and 60%, between about 10% and 80%, between about 15% and 25%, between about 15% and 30%, between about 15% and 40%, between about 15% and 50%, between about 15% and 60%, between about 15% and 80%, between about 20% and 30%, between about 20% and 40%, between about 20% and 50%, between about 20% and 60%, between about 20% and 80%, between about 30% and 50%, between about 30% and 60%, between about 30% and 80%, between about 40 and 60%, between about 40% and 80%, or between about 50% and 80% AT1 cells. Similarly, the lung forming progenitor cells can be coadministered with between about 1% and 5%, between about 1% and 10%, between about 1% and 15%, between about 1% and 20%, between about 1% and 25%, between about 1% and 30%, between about 1% and 40%, between about 1% and 50%, between about 1% and 60%, between about 1% and 80%, between about 5% and 10%, between about 5% and 15%, between about 5% and 20%, between about 5% and 25%, between about 5% and 30%, between about 5% and 40%, between about 5% and 50%, between about 5% and 60%, between about 5% and 80%, between 10% and 20%, between about 10% and 25%, between about 10% and 30%, between about 10% and 40%, between about 10% and 50%, between about 10% and 60%, between about 10% and 80%, between about 15% and 25%, between about 15% and 30%, between about 15% and 40%, between about 15% and 50%, between about 15% and 60%, between about 15% and 80%, between about 20% and 30%, between about 20% and 40%, between about 20% and 50%, between about 20% and 60%, between about 20% and 80%, between about 30% and 50%, between about 30% and 60%, between about 30% and 80%, between about 40 and 60%, between about 40% and 80%, or between about 50% and 80% AT2 cells. Additionally, the lung forming progenitor cells can be coadministered with between about 1% and 5%, between about 1% and 10%, between about 1% and 15%, between about 1% and 20%, between about 1% and 25%, between about 1% and 30%, between about 1% and 40%, between about 1% and 50%, between about 1% and 60%, between about 1% and 80%, between about 5% and 10%, between about 5% and 15%, between about 5% and 20%, between about 5% and 25%, between about 5% and 30%, between about 5% and 40%, between about 5% and 50%, between about 5% and 60%, between about 5% and 80%, between 10% and 20%, between about 10% and 25%, between about 10% and 30%, between about 10% and 40%, between about 10% and 50%, between about 10% and 60%, between about 10% and 80%, between about 15% and 25%, between about 15% and 30%, between about 15% and 40%, between about 15% and 50%, between about 15% and 60%, between about 15% and 80%, between about 20% and 30%, between about 20% and 40%, between about 20% and 50%, between about 20% and 60%, between about 20% and 80%, between about 30% and 50%, between about 30% and 60%, between about 30% and 80%, between about 40 and 60%, between about 40% and 80%, or between about 50% and 80% endothelial cells. The lung forming progenitor cells can also be coadministered with between about 1% and 5%, between about 1% and 10%, between about 1% and 15%, between about 1% and 20%, between about 1% and 25%, between about 1% and 30%, between about 1% and 40%, between about 1% and 50%, between about 1% and 60%, between about 1% and 80%, between about 5% and 10%, between about 5% and 15%, between about 5% and 20%, between about 5% and 25%, between about 5% and 30%, between about 5% and 40%, between about 5% and 50%, between about 5% and 60%, between about 5% and 80%, between 10% and 20%, between about 10% and 25%, between about 10% and 30%, between about 10% and 40%, between about 10% and 50%, between about 10% and 60%, between about 10% and 80%, between about 15% and 25%, between about 15% and 30%, between about 15% and 40%, between about 15% and 50%, between about 15% and 60%, between about 15% and 80%, between about 20% and 30%, between about 20% and 40%, between about 20% and 50%, between about 20% and 60%, between about 20% and 80%, between about 30% and 50%, between about 30% and 60%, between about 30% and 80%, between about 40 and 60%, between about 40% and 80%, or between about 50% and 80% mesenchymal cells. The lung forming progenitor cells can be coadministered with a combination of AT1, AT2, endothelial, and mesenchymal cells. For example, the lung forming progenitor cells can be coadministered with between about 1% and 5%, between about 1% and 10%, between about 1% and 15%, between about 1% and 20%, between about 1% and 25%, between about 1% and 30%, between about 1% and 40%, between about 1% and 50%, between about 1% and 60%, between about 1% and 80%, between about 5% and 10%, between about 5% and 15%, between about 5% and 20%, between about 5% and 25%, between about 5% and 30%, between about 5% and 40%, between about 5% and 50%, between about 5% and 60%, between about 5% and 80%, between 10% and 20%, between about 10% and 25%, between about 10% and 30%, between about 10% and 40%, between about 10% and 50%, between about 10% and 60%, between about 10% and 80%, between about 15% and 25%, between about 15% and 30%, between about 15% and 40%, between about 15% and 50%, between about 15% and 60%, between about 15% and 80%, between about 20% and 30%, between about 20% and 40%, between about 20% and 50%, between about 20% and 60%, between about 20% and 80%, between about 30% and 50%, between about 30% and 60%, between about 30% and 80%, between about 40 and 60%, between about 40% and 80%, or between about 50% and 80% cells selected from AT1, AT2, endothelial, or mesenchymal cells. Alternatively, instead of lung forming progenitor cells, a method disclosed herein can include administering cells selected from AT1, AT2, endothelial, or mesenchymal cells.

In some aspects, the lung forming progenitor cells include CD45⁺ hematopoietic cells. For example, the lung forming progenitor cells can include between about 1% and 5%, between about 1% and 10%, between about 1% and 15%, between about 1% and 20%, between about 1% and 25%, between about 1% and 30%, between about 1% and 40%, between about 1% and 50%, between about 1% and 60%, between about 1% and 80%, between about 5% and 10%, between about 5% and 15%, between about 5% and 20%, between about 5% and 25%, between about 5% and 30%, between about 5% and 40%, between about 5% and 50%, between about 5% and 60%, between about 5% and 80%, between 10% and 20%, between about 10% and 25%, between about 10% and 30%, between about 10% and 40%, between about 10% and 50%, between about 10% and 60%, between about 10% and 80%, between about 15% and 25%, between about 15% and 30%, between about 15% and 40%, between about 15% and 50%, between about 15% and 60%, between about 15% and 80%, between about 20% and 30%, between about 20% and 40%, between about 20% and 50%, between about 20% and 60%, between about 20% and 80%, between about 30% and 50%, between about 30% and 60%, between about 30% and 80%, between about 40 and 60%, between about 40% and 80%, or between about 50% and 80% CD45⁺ hematopoietic cells. Similarly, CD45⁺ hematopoietic cells can constitute between about 1% and 5%, between about 1% and 10%, between about 1% and 15%, between about 1% and 20%, between about 1% and 25%, between about 1% and 30%, between about 1% and 40%, between about 1% and 50%, between about 1% and 60%, between about 1% and 80%, between about 5% and 10%, between about 5% and 15%, between about 5% and 20%, between about 5% and 25%, between about 5% and 30%, between about 5% and 40%, between about 5% and 50%, between about 5% and 60%, between about 5% and 80%, between 10% and 20%, between about 10% and 25%, between about 10% and 30%, between about 10% and 40%, between about 10% and 50%, between about 10% and 60%, between about 10% and 80%, between about 15% and 25%, between about 15% and 30%, between about 15% and 40%, between about 15% and 50%, between about 15% and 60%, between about 15% and 80%, between about 20% and 30%, between about 20% and 40%, between about 20% and 50%, between about 20% and 60%, between about 20% and 80%, between about 30% and 50%, between about 30% and 60%, between about 30% and 80%, between about 40 and 60%, between about 40% and 80%, or between about 50% and 80% of all cells administered to the subject.

In particular aspects, the lung forming progenitor cells include AT2 cells. Notably, it was demonstrated herein that in IPF patients, a subclinical injury of the epithelium is associated with AT2 cell attrition and the presence of a subset of dysfunctional AT2 cells, and furthermore that AT2 cells in IPF exhibit a pro-fibrotic phenotype that likely activates fibroblasts, mesenchymal cell expansion and ECM deposition. Without being bound by theory, it is contemplated herein that these changes are associated with the failure of AT2 cells to differentiate into AT1 alveolar cells and the disruption of the epithelial-mesenchymal interface. Furthermore, AT2 cell erosion may lead to opportunistic expansion of fibroblasts, further decreasing the gas exchange properties of the lung surface.

In some cases, the progenitor cell treatment efficacy can be enhanced by targeted depletion or enrichment. For example, in various aspects, the method includes enriching the cells for those expressing an endothelial marker and/or an epithelial marker and/or depleting the cells of cells expressing CD45.

In some aspects, the method includes enriching the cells for those expressing an epithelial marker. Epithelial cell populations often exhibit depletion and inhibition in fibrotic lungs. In particular, profibrotic conditions can induce epithelial-mesenchymal transitions within epithelial populations, adversely affecting epithelial (e.g., alveolar) architecture, and possibly inhibiting epithelial repair. Epithelial progenitor cell administration can replenish lung epithelial populations to improve lung function and inhibit fibrosis. In some aspects, the epithelial marker is selected from CD326, CD324, CD245, aquaporin-5, podoplanin, or advanced glycosylation end-product specific receptor. In some aspects, the epithelial marker is CD326.

In some aspects, the method includes enriching the cells for those expressing an endothelial marker. Endothelial progenitor cell administration can reestablish antifibrotic homeostasis in fibrotic lungs. Fibrosis, inflammation, and endothelial damage can induce resident lung endothelial cells to produce profibrotic and epithelial repair inhibitory factors, thereby furthering fibrotic progression. Reestablishing healthy endothelial lung tissue not only can reverse fibrosis, but can enhance lung repair. In certain aspects, the endothelial marker is CD31 or CD144.

In some aspects, the method includes enriching the cells for those expressing at least two markers. In some aspects, the at least two markers are selected from CD31⁺, CD144⁺, CD324⁺, and CD326⁺. In some aspects, the method further includes enriching the cells for those that are CD326⁺CD31⁺, CD324⁺CD31⁺, CD326⁺CD144⁺, and/or CD324⁺CD144⁺.

Advantageously, pulmonary tissue can contain a sufficient density of patch forming cells so as to not require enrichment prior to administration. In some aspects, the method includes determining that the cells include a cell which is positive for an endothelial marker and an epithelial marker. In some aspects, the method includes determining that the cells include a CD326⁺CD31⁺, CD324⁺CD31⁺, CD326⁺CD144⁺, and/or CD324⁺CD144⁺ cell. In some aspects, the method does not include enrichment.

In some aspects, the method further comprises determining expression of an epithelial marker, an endothelial marker, or a combination thereof in the cells. As non-limiting examples, the epithelial marker can be selected from CD326, CD324, CD245, aquaporin-5, podoplanin, or advanced glycosylation end-product specific receptor. In some cases, the epithelial marker is CD326. In some cases, the endothelial marker is CD31, CD144, or ERG. In some cases, the endothelial marker is CD31. In some cases, the epithelial marker is CD326 and the endothelial marker is CD31. In some cases, a predetermined proportion of the cells express the epithelial marker and the endothelial marker. For example, at least about 0.1%, at least about 1%, at least about 2%, at least about 5%, at least about 10%, at least about 20%, at least about 30%, at least about 40%, at least about 50%, at least about 60%, at least about 70%, at least about 80%, at least about 90%, or at least about 95% of the cells may express the epithelial marker and the endothelial marker. In some cases, the cells are expanded for at least about 12 hours, at least about 24 hours, at least about 36 hours, at least about 48 hours, at least about 72 hours, at least about 120 hours, at least about 180 hours, at least about 240 hours, at least about 300 hours, least about 600 hours, or at least about 1200 hours prior to the determining. In some cases, the cells are expanded for at least about 12 hours, at least about 24 hours, at least about 36 hours, at least about 48 hours, at least about 72 hours, at least about 120 hours, at least about 180 hours, at least about 240 hours, at least about 300 hours, or at least about 600 hours, or at least about 1200 hours subsequent to the determining.

In some aspects, the cells are determined to not be suitable for administration to the subject when a proportion of the cells which do not express the epithelial marker and the endothelial marker exceeds a predetermined threshold. The predetermined threshold can be at least about 0.1%, at least about 1%, at least about 2%, at least about 5%, at least about 10%, at least about 20%, at least about 30%, at least about 40%, at least about 50%, at least about 60%, at least about 70%, at least about 80%, at least about 90%, or at least about 95% of the cells. In such cases, endothelial and epithelial-positive cells can be enriched from the sample, or a new sample may be collected and analyzed for endothelial and epithelial-positive cell-content.

In certain aspects, the method further includes depleting the cells of T cells, depleting the cells of B cells, or a combination thereof. T cell depletion can prevent graft vs host disease (GVHD), as well as common GVHD-associated symptoms including nausea, ulceration, and skin-discoloration. T and B cell depletion can also limit B cell responses which can otherwise induce Epstein-Barr virus-associated proliferative diseases. In such aspects, the method can include depleting CD19⁺, CD20⁺, CD45⁺, CD4⁺, CD8⁺, CD127⁺, PD-1⁺, CD122⁺, and/or CD132⁺ cells. In one aspect, the method includes depleting CD45⁺ cells.

In one aspect, enriching the cells or depleting the cells includes contacting the cells with an agent that binds to the epithelial marker, the endothelial marker, or a T cell marker. In one aspect, enriching the cells or depleting the cells includes contacting the cells with an agent that binds to the epithelial marker, the endothelial marker, or CD45. In another aspect, the binding agent is an antibody. In some aspects, the antibody is coupled to a substrate.

In some aspects, the method further includes expanding the isolated pulmonary cells in culture. The isolated pulmonary cells can be cultured with suitable media, antibiotics, growth factors, nutrients (e.g., amino acids) conducive to cellular expansion. The expansion can enrich specific types of cells, for example epithelial, endothelial, or mesenchymal progenitor cells.

In various aspects, the subject has idiopathic pulmonary fibrosis or chronic obstructive pulmonary disease. In some aspects, the idiopathic pulmonary fibrosis or chronic obstructive pulmonary disease includes honeycombing, ground-glass opacities, reticular opacities, basal predominant reticulation, traction bronchiectasis, or a combination thereof. In some aspects, the idiopathic pulmonary fibrosis or chronic obstructive pulmonary disease includes senescence.

In certain aspects, the cells are administered in one or more dose. In some aspects, the subject is administered a single dose of lung forming progenitor cells. In some aspects, the subject is administered multiple doses of lung forming progenitor cells. The doses can be identical, or can differ in form, excipient-type, and in the number and type of lung forming progenitor cells. In some aspects, identical doses of the lung forming progenitor cells are administered to the subject at least 2, at least 3, at least 4, at least 5, at least 6, at least 7, at least 8, at least 9, or at least 10 times. In some aspects, the subject is administered doses of the lung forming progenitor cells until their fibrosis is ameliorated or inhibited. For example, the subject may be administered doses of the lung forming progenitor cells in regular intervals until their lung fibrosis falls below a certain threshold. In some aspects, the method includes repeating the administering every 1 to 400 days. For example, a single dose of the lung forming progenitor cells can be administered to the subject on a daily, weekly, biweekly, monthly, bimonthly, semiannual, or annual basis.

In some aspects, the subject is administered between about 5×10² and 5×10⁷ lung forming progenitor cells. In some aspects, the subject is administered between about 5×10² and 5×10⁵, between about 5×10³ and 5×10⁶, or between about 5×10⁴ and 5×10⁷ lung forming progenitor cells. In additional aspects, a composition that includes the lung forming progenitor cells includes between 5 million/Kg to 500 million/Kg lung cells. In further aspects, CD326⁺CD31⁺ lung progenitor cells include between about 0.1% and 1%, between about 0.1% and 2%, between about 0.1% and 3%, between about 0.1% and 4%, between about 0.1% and 5%, between about 0.1% and 6%, between about 0.1% and 8%, between about 0.5% and 1%, between about 0.5% and 2%, between about 0.5% and 3%, between about 0.5% and 4%, between about 0.5% and 5%, between about 0.5% and 6%, between about 0.5% and 8%, between about 1% and 2%, between about 1% and 3%, between about 1% and 4%, between about 1% and 5%, between about 1% and 6%, between about 1% and 8 %, between about 2% and 3%, between about 2% and 4%, between about 2% and 5%, between about 2% and 6%, between about 2% and 8%, between about 3% and 4%, between about 3% and 5%, between about 3% and 6%, between about 3% and 8%, between about 4% and 5%, between about 4% and 6%, between about 4% and 8%, between about 5% and 6%, between about 5% and 8%, or between about 6% and 8% CD326⁺CD31⁺ lung progenitor cells.

In one embodiment, the invention provides a method of improving lung function in a subject in need thereof which includes administering to a subject having at least about 15% lung fibrosis a therapeutically effective amount of lung forming progenitor cells, thereby improving lung function in the subject. As demonstrated herein, in addition to remediating fibrosis, lung forming progenitor cell treatment can improve lung function, including forced expired volume capacity and lung resistance, promote wound healing, and replace populations of host-derived patch-forming cells. In many aspects, the administering is performed in the absence of a pre-conditioning treatment prior to the administration of the cells. Accordingly, the method can provide a mild treatment alternative to cell therapy treatments which require damage-inducing pre-conditioning regimens. In some aspects, the lung forming progenitor cells increase a number of donor-derived patch forming cells. In some aspects, the lung forming progenitor cells decrease a number of host-derived patch-forming cells.

In one aspect, prior to administering, a level of fibrosis in the lung of the subject is measured by comparing a fibrosis volume in the lung to a total lung volume. In some aspects, fibrosis volume is parenchymal fibrosis volume (e.g., as detailed in Ikezoe et al. American Journal of Respiratory and Critical Care Medicine 2021; 204(9): 1045-59.). In some aspects, fibrosis volume and total lung volume are determined with a CT scan and/or lung biopsy analysis.

In another aspect, improving lung function includes reducing fibrosis and/or preventing or slowing down fibrosis progression. In some aspects, improving lung function includes improving respiratory system Newtonian resistance, lung tissue compliance, lung tissue elastance, tissue dampening and/or forced expired volume. For example, the subject may exhibit improved performance in a pulmonary mechanics test.

In certain aspects, the subject has pulmonary fibrosis or chronic obstructive pulmonary disease. In some aspects, the idiopathic pulmonary fibrosis or chronic obstructive pulmonary disease includes honeycombing, ground-glass opacities, reticular opacities, basal predominant reticulation, traction bronchiectasis, or a combination thereof. In some cases, the subject has at least about 15%, at least about 16%, at least about 17%, at least about 18%, at least about 19%, at least about 20% lung fibrosis, at least about 21% lung fibrosis, at least about 22% lung fibrosis, at least about 23% lung fibrosis, at least about 24% lung fibrosis, or at least about 25% lung fibrosis.

In some aspects, the cells are administered in a cell suspension. The cell suspension can include a consortia of lung cells including the lung forming progenitor cells. Alternatively, the lung forming progenitor cells can constitute the majority of cells within the suspension. For example, in some aspects, at least about 50%, at least about 60%, at least about 70%, at least about 80%, at least about 85%, at least about 90%, at least about 95%, at least about 98%, or at least about 99% of cells within the cell suspension are lung forming progenitor cells. In some cases, less than about 20%, less than about 15%, less than about 12%, less than about 10%, less than about 8%, less than about 6%, less than about 4%, less than about 2%, or less than about 1% of cells in the cell suspension are immune cells. The cell suspension can include between about 5×10² and 5×10⁷, between about 5×10² and 5×10⁵, between about 5×10³ and 5×10⁶, or between about 5×10⁴ and 5×10⁷ cells or between 5×10⁷ to 1×10⁹ cells. Alternatively, or in addition thereto, the cell suspension can include between about 5×10² and 5×10⁷, between about 5×10² and 5×10⁵, between about 5×10³ and 5×10⁶, or between about 5×10⁴ and 5×10⁷ lung forming progenitor cells.

The cell suspension can include a therapeutically effective amount of the lung forming progenitor cells. Ass used herein, the term “effective amount” of an active agent refers an amount that is non-toxic to a subject but is an amount of the active agent that is sufficient to provide a desired effect (e.g., treatment of a skeletal muscle disorder, metabolic disorder, blood disorder, or cancer). This amount may vary from subject to subject, depending on the species, age, and physical condition of the subject, the severity of the disease that is being treated, the particular conjugate, or more specifically, the particular active agent used, its mode of administration, and the like. Therefore, it is difficult to generalize an exact “effective amount,” yet, a suitable effective amount may be determined by one of ordinary skill in the art.

The terms “therapeutically effective amount”, “effective dose,” “therapeutically effective dose”, “effective amount,” or the like refer to the number of lung forming progenitor cells that will elicit the biological or medical response of a tissue, system, animal or human that is being sought by the researcher, veterinarian, medical doctor or other clinician. Generally, the response is either amelioration of symptoms in a patient or a desired biological outcome (e.g., the prevention or amelioration of fibrosis). Such an amount should be sufficient to inhibit or treat fibrosis, and can be determined as described herein In one embodiment, the invention provides a method of identifying a subject with pulmonary fibrosis as a candidate for treatment with lung forming progenitor cells which includes a) measuring the level of fibrosis in the lungs of the subject; and i) classifying a subject with a level of fibrosis of at least about 15% or greater as a likely responder to cell transplantation, thereby identifying the subject as suitable for cell transplantation, or ii) classifying a subject with a level of fibrosis of about 0 to less than 15% as a likely non-responder to lung cell transplantation, thereby identifying the subject as unsuitable for cell transplantation, thereby identifying a subject with pulmonary fibrosis as a candidate for treatment with lung forming progenitor cells.

In one aspect, measuring the level of fibrosis is by a computed tomography (CT) scan and/or assessing the level of fibrosis on a lung biopsy sample. In some aspects, the level of fibrosis in the lung of the subject is measured by comparing a fibrosis volume in the lung to a total lung volume. In some aspects, fibrosis volume is parenchymal fibrosis volume. In some aspects, the fibrosis is identified as honeycombing, ground-glass opacities, reticular opacities, and combinations thereof.

In a further aspect, the method further includes administering a cell suspension with isolated lung forming progenitor cells to the subject identified as a likely responder. In some aspects, administering the cell suspension includes increasing a number of donor-derived patch forming cells and/or decreasing a number of host-derived patch-forming cells. For example, in some cases, donor cell engraftment results in a decreased number of host-derived patch-forming cells in the subject. In certain aspects, the donor-derived patch forming cells express both endothelial and epithelial markers. In some aspects, the endothelial marker is CD31, CD144, or ERG. In some aspects, the endothelial marker is CD31. In some aspects, the epithelial marker is CD326. In some aspects, the epithelial marker is selected from CD326, CD324, CD245, aquaporin-5, podoplanin, or advanced glycosylation end-product specific receptor. In some aspects, the epithelial marker is CD326.

In some aspects, the method further includes measuring a second level of fibrosis in the lungs of the subject at a second time point and (i) maintaining the subject as an unlikely responder if the second level of fibrosis is of about 0 to less than 15%; or (ii) reclassifying the subject as a likely responder if the second level of fibrosis is of at least about 15% or greater. In some aspects, a level of fibrosis of at least about 15% or greater is indicative of a reduction in a number of endogenous lung stem cells sufficient to ensure engraftment of the lung forming progenitor cells. In some aspects, the subject is administered a cell suspension with isolated lung forming progenitor cells following measuring of the second level of fibrosis in the lungs of the subject (e.g., following reclassification of the subject as a likely responder to lung forming progenitor cell treatment). In certain aspects, a level of fibrosis of about 1 to less than 15% is indicative of an absence of a sufficient reduction in a number of endogenous lung stem cells to allow engraftment of the lung forming progenitor cells. In some aspects, the second time point is 10 to 1000 days after the initial instance of measuring the level of fibrosis in the lungs of the subject. In some aspects, the second time point is 50 to 500 days after the initial instance of measuring the level of fibrosis in the lungs of the subject.

Presented below are examples discussing lung forming progenitor cell therapies contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Materials and Methods

### Mice

Animals were maintained under conditions approved by the Institutional Animal Care and Use Committee at MD Anderson (protocols no. 1976, 1819). Mouse strains used included: C57BL/6, C57BL/6-TRF1 (B6-129P2-Terf1^{tm2.1Td1}) and B6-SPC-Cre (B6.129S-Sftpc^{tm1(cre-ERT2)B1h}), as well as donor mice for transplantation experiments including C57BL/6-Tg (CAG-EGFP)1Osb/J and B6.129(Cg)-Gt(ROSA)26Sor^{tm4(ACTB-tdTomato,-EGFP)Luo}/J. These latter mice express GFP or TdTomato, respectively, in all lung cells. All mice were used at 6-30 weeks of age. Mice were kept in small cages (up to five animals in each cage) and fed sterile food and acid water. Animals of the same age, sex, and genetic background were randomly assigned to treatment groups. Pre-established exclusion criteria were based on IACUC guidelines, and included systemic disease, respiratory distress, refusal to eat and drink, and substantial (>15%) weight loss. For BLM experiments, only male mice were used as hosts because male mice develop lung fibrosis to a greater degree than female mice. For experiments using the SPC-Cre TRF1A/f model, mice of both genders were used as hosts. A minimum of 5 mice per group were used in all experiments.

### SPC-Cre TRF1^{fl/fl} Genetic Model

C57BL/6-TRF1 (B6-129P2-Terf1tm2.1Td1) were received from Jax labs after cryo-recovery, and backcrossed in lab with C57BL/6 for four generations in order to ensure a clean C57BL/6 background. Mice homozygous for the Lox insertion were mated with transgenic mice that express Cre-ERT protein under the control of surfactant promoter (SFTPC)-B6-SPC-Cre (B6.129S-Sftpctm1(cre-ERT2) Blh). The first generation of heterozygous B6-TRFWT/Lox SPC-CreERT mice were backcrossed between them until a strain of B6-TRFLox/Lox SPC-CreERT mice was attained. To activate the Cre protein, mice were injected I.P. with 50 mg of tamoxifen per kg (TMX, Sigma) for 6-7 weeks, 3 injections per week. TMX was dissolved in corn oil to a concentration of 10 μg/μl freshly before each administration.

### Induction of Fibrosis by Bleomycin

C57BL/6 male mice aged 9-10 weeks were administered 0.035 U/gr bleomycin (Hikma or Teva) by i.p. injection twice a week for 1 to 5 weeks. Control groups were administered PBS vehicle. Mice were weighed twice a week and those that lost weight were fed with extra high fat food. Weight loss of more than 20% led to euthanizing the mice.

### Pre-Conditioning of Host C57BL/6 Mice Prior to Transplantation

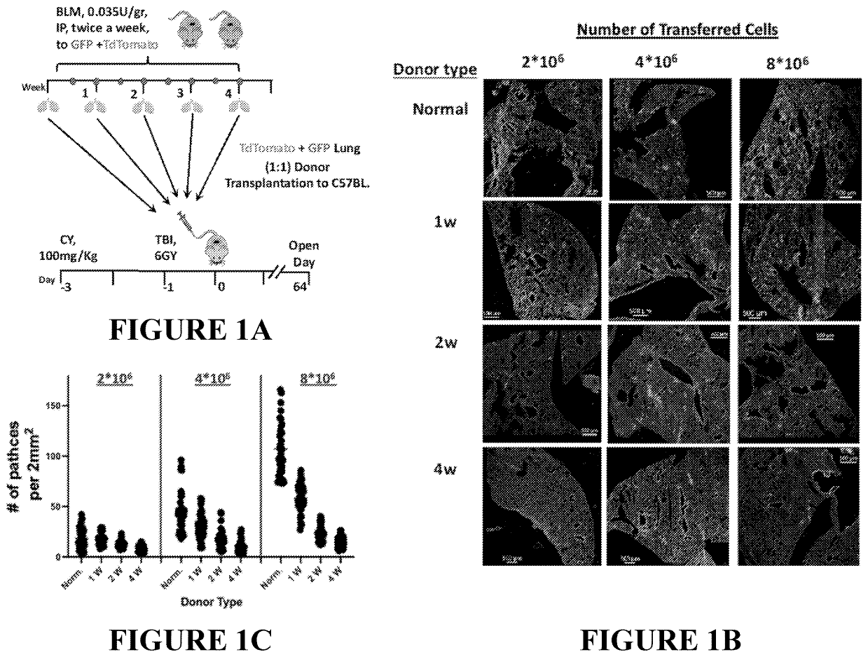

An assay that we previously described was used to test the level of patch forming lung progenitors after BLM treatment (Milman et al., Stem Cells Transl Med, 2022; 11(2): 178-188), based on conditioning of recipient mice with CY and 6 Gy TBI. On day −3 prior to transplantation, the C57BL/6 host mice were treated with i.p. injection of 200 mg/kg CY (Sandoz or Baxter) dissolved in PBS. On day −1 before transplantation, the mice were treated with 6 Gy TBI in an Xrad-320 biological X-ray irradiator. On day 0, the mice were transplanted with various cell populations, as indicated in FIG. 1.

### Preparation of Single Cell Lung Suspension

Single cell suspensions were obtained from enzymatically treated adult and fetal mouse lungs. Briefly, lung tissue was dissociated by mincing tissues fine scissors in the presence of 1 mg/ml collagenase, 2.4 U/ml dispase and 1 mg/ml DNAseI (Roche Diagnostics) diluted in Ca+Mg+ phosphate buffered saline. Cells were dissociated by GentalMax (Miltenyi Biotec) incubation for 30 minutes at 370 C. Nonspecific debris was removed by sequential filtration through 100 and 70 μm filters. The cells were then washed with PBS (Ca²⁺ and Mg²⁺ free) with 2% bovine serum albumin (BSA), antibiotics and 2 mM Anticoagulant Citrate Dextrose Solution A. Before I.V. injection, the donor cells were filtered again through a 40 um filter. Host mice were transplanted with 2-8×10⁶ adult lung cells, either B6-GFP or B6-TdTomato. Transplanted cells were introduced by intravenous cell injection (I.V.) into the tail vein.

### Flow Cytometry

Cell samples were stained with conjugated antibodies or matching isotype controls according to the antibody manufacturer's instructions. Antibodies were purchased from e-Bioscience and Biolegend. Data were acquired on BD FACSCanto II or BD LSRFortessa flow cytometer, and analyzed using FlowJo software (version 9, or version 10).

Immunostaining Mice were sacrificed at different time points following transplantation; the lungs were inflated to full capacity with 4% paraformaldehyde (PFA) solution introduced through the trachea under a constant pressure of 20 cm H₂O. Then, the lungs were immersed in fixative overnight at 4° C. The next day the lungs were split to two halves-one half was preserved in 30% sucrose for an additional 24 hours prior to snap freezing in isopentane pre-cooled by liquid nitrogen in the presence of Optimal Cutting Temperature (OCT) compound (Sakura Finetek USA, Inc. Tissue-Tek.; product code #4583). The second half was preserved with 70% ethanol before paraffin embedding. The samples for frozen sections were harvested and inflated with a 1:1 mixture of OCT and PBS and snap frozen in isopentane pre-cooled by liquid nitrogen. Frozen samples were cut to 6-12 um sections and stained. All secondary antibodies were purchased from Jackson Laboratories or Abcam. Evaluation of stained samples was performed by upright Olympus BX51 fluorescent microscope with ×4, ×10, ×20, and ×40 air and ×100 oil objectives, and Olympus digital camera (DP70). Confocal microscopy was performed on an Olympus 3000FV laser scanning confocal microscope, using cell sense software (Olympus). The images were processed, rendered and reconstructed in 3D in Imaris software (Bitplane AG, Switzerland, www. bitplane. com).

### Tri-Chrome Staining and Ashcroft Test for Fibrosis Assessment

The lower-mid part of the right lungs, which were fixed by PFA, was deparaffinized and stained with Masson's trichrome using Masson Trichrome Staining Kit (Thomas Scientific) according to the manufacturer's instructions. More than 20 bright field images per mouse were taken, using 20-fold magnification. The fields were chosen randomly by moving the stage controls of the microscope blindly and photographing the resulting field without further stage adjustments. Every field imaged had at least 80% covered with lung tissue. Sections were chosen to ensure it represents all grade of fibrosis. The image files were name coded and were given to 3 different observers, blinded to the experimental groups. The observers were provided with a copy of the scoring scheme and a set of different grade examples. Each observer scored all the images from 0 to 8 using the specific criteria described in Ashcroft et al., J Clin Pathol., 1988; 41(4): 467-70 and Hubner et al., Biotechniques, 2008; 44(4): 507-17. All scores were then pooled, and the average was calculated per field and per mouse, and then per group with different treatments. The results were also divided into three scoring bins: a score of 0-3 for normal lung tissue, a score of 4-5 for mild damage, a score of 6-8 for severe fibrotic damage. Results were analyzed using PRISM software. Large field images were taken using ImageXpress Micro Confocal.

### CT Scan and Calculation of Fibrotic Area

In vivo micro-CT scan were performed by high resolution Skyscan 1276 (Bruker BioSpin corporation, MA) at 30 um resolution, 135 msec exposure time, 0.5 mm filter and with the use of 1008×672 matrix. Mice were anesthetized at 0.5% oxygen and 1.5% isoflurane. 3D images and fibrosis percentage calculations were performed using 55/255 threshold. Lung volume (LV) and fibrosis volume (FV) were measured. The percent fibrosis (FV/LV) was calculated as the percent of fibrosis volume out of total lung volume.

### Quantitative Assay for Total Collagen Levels

To evaluate total collagen levels, we used an assay based on a colorimetric read-out for free hydroxyproline. Briefly, paraffin-embedded lung samples were weighed and hydrolysed overnight and then assessed by the color reaction for the amount of collagen using the QuickZyme kit (QuickZyme, Biosciences, Netherlands) according to the manufacturer's instructions.

### Assessment of Lung Function

Respiratory system mechanics and P-V relationships were measured using Flexi Vent apparatus (SCIREQ, Montreal, QC, Canada). In brief, after mice were anesthetized using Avertin, tracheotomized using 19 Gauge metal cannula (Brico) and connected to the FlexiVent via endotracheal cannula, they were ventilated at a respiratory rate of 150 breaths/min and tidal volume of 10 ml/Kg against positive end-expiratory pressure of 3 cm H₂O. FlexiVent software were used to perform the forced measurements and P-V loops.

The linear single frequency forced oscillation technique (FOT) was used to assess total respiratory system resistance (R), compliance (C) and elastance (E). The broadband FOT was used to determine Newtonian resistance (Rn), tissue elastance (H) and tissue dampening (G). Volume-driven P-V loops were formed from incrementally inflating the lungs to 40 ml kg⁻¹ from functional residual capacity, which was defined as 3 cm H₂O. After the delivery of each volume increment, the airway opening pressure was recorded. The area of the P-V curve was calculated using flexiVent software to provide data for quantitative analysis of the elastic properties. All measurements of respiratory system mechanics were conducted in mice with intact chest walls. Upon completion of the measurements, anaesthetized animals were killed by cervical dislocation. Results were analyzed using PRISM software.

### Hyaluronan (HA) and Alpha-SMA Double Staining

Deparaffinized lung sections were stained with biotinylated HA binding protein (HABP) (Vector laboratories, US) after blocking with normal horse serum (2.5%, Vector Laboratories). After washing, the sections were stained in blue using substrate for alkaline phosphatase (AP) enzyme (Vector Blue kit). For alpha SMA staining sections were blocked again, washed and incubated overnight with the alpha SMA (Thermo fisher, US) primary antibody following treatment with the ImmPact detection kits for alkaline phosphatase (Vector laboratories) to stain the alpha SMA in red according to the manufacturer's instructions.

### Statistical Analysis

Differences between groups were evaluated using one-way variance analysis (ANOVA) and Dunnett's post-hoc test for calculating the p value between the 3 or more different groups, or unpaired T-test for comparison of 2 groups only, using Prism software. For each data set, mean±SD or mean±SEM was calculated, and is presented in the Results section of the main text. P value≤0.03 was considered statistically significant.

### Example 2

### Time Window for Successful Engraftment of Donor Derived Epithelial and Endothelial Cells in the BLM Mouse Model

In NA (naphthalene) or CY (cyclophosphamide) treated mice, engraftment and colonization of lung forming progenitors was shown to require subsequent treatment two days later with sublethal 6 GY total body irradiation (TBI). This pre-transplant conditioning enables to effectively vacate endogenous lung forming progenitor cells from their respective niches, thereby reducing stem cell competition between host and donor cells for these niches.

In the present study the therapeutic efficacy of this transplantation modality was evaluated for lung fibrosis. To that end two distinct mouse models, namely, the bleomycin (BLM) and the TRF1/SPC models were used.

BLM induces fibrotic reaction in mice within a short period (1-3 weeks post administration) with some strain variability; C57BL/6 and Balb/c mice exhibit high and low and fibrotic damage, respectively. Also, females exhibit a higher resistant to BLM associated with production of anti-proteases damage proteins, which are X linked. Notably, it has been suggested that BLM induced fibrosis is partially reversible and therefore its use was largely limited to short term experiments, assessing potential therapeutic agents within 4-5 weeks after completion of BLM administration. Bleomycin is generally administrated intratracheally (IT) using a single dose that mostly produces a bronchiolocentric distribution of fibrosis. Alternatively, it can be administered intravenously (IV) or intraperitoneally (IP) both of which induce mostly subpleural scarring of the lung tissue, as commonly observed in IPF patients. Chronic IP administration of bleomycin leads to more persistent and less reversible fibrosis. Thus, the protocol used relied on low dose of BLM administered over a period of several weeks in order to induce more stable lung fibrosis which enables to assess lung stem cell transplantation modality which requires 6-8 weeks for attaining substantial level of donor derived lung cell chimerism.

In NA or CY treated mice, engraftment and colonization of lung progenitors requires subsequent conditioning treatment after 2 days with sublethal 6 GY TBI. This pre-transplantation conditioning enables endogenous lung progenitors to be effectively vacated from their niches, thereby reducing stem cell competition between host and donor cells for these niches. Thus, the impact of BLM treatment on endogenous patch-forming cells was initially interrogated. To that end, a transplantation assay in which recipient mice are conditioned by CY and 6 GY TBI was employed. C57BL/6- Tdtomato^{+ (B}6.129(Cg)-Gt(ROSA)26Sor^{tm4(ACTB-tdTomato,-EGFP)Luo}/J) and C57BL/6- GFP⁺ (C57BL/6-Tg (CAG-EGFP)1Osb/J) mice (Green Fluorescent Protein), used as donors, were treated with BLM for 1, 2 or 4 weeks, and thereafter, lung cells were harvested and transplanted in different doses (2, 4, or 8×10⁶ cells) into recipient C57BL/6 mice conditioned with CY and 6 Gy TBI (FIG. 1A). Donor-derived patches were determined in recipient mice at 64 days post-transplant. Considering that the TdTomato and GFP donor mice were congenic to the recipient animals and might be rejected to some extent, a 1:1 mixture of cells from both strains was used to ascertain that strong engraftment is exhibited after transplantation of lung cells from both types of donors. Also, due to limited availability of fluorescent donor mice, this approach was useful for attaining the large number of fluorescent donor cells required for the extensive transplantation experiments.

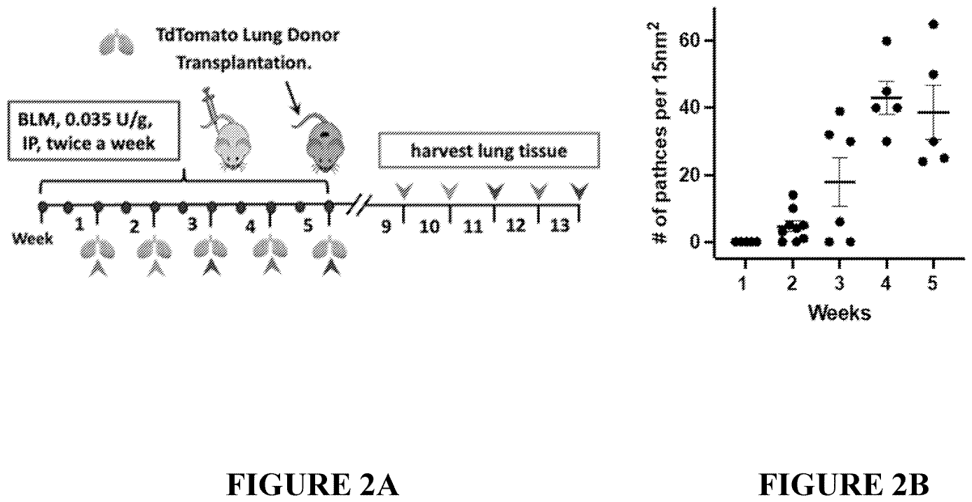

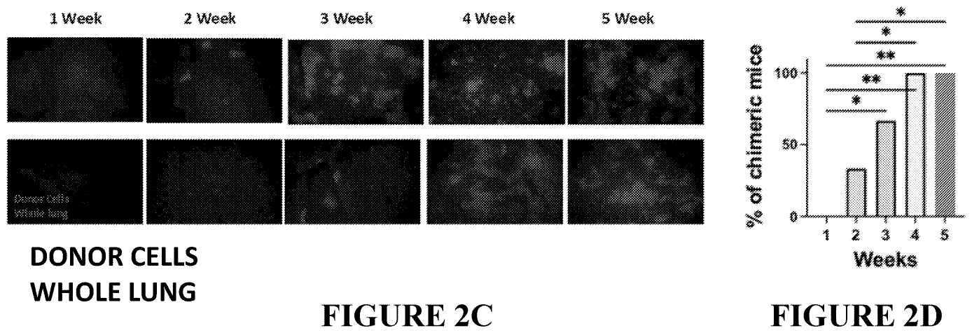

As shown in FIGS. 2B-2C depicting typical immunohistology of donor derived patches and quantitative analysis of patch number per 2 mm² lung tissue, respectively, high levels of patch-forming progenitors persisted in donor mice following BLM treatment for 1-2 weeks, while 4 weeks of BLM treatment markedly reduced the patch-forming lung cells. These results strongly suggest that BLM treatment alone is associated with loss of endogenous patch-forming lung progenitors, and can make space for engraftment of donor-derived patch forming cells when used in recipient animals.

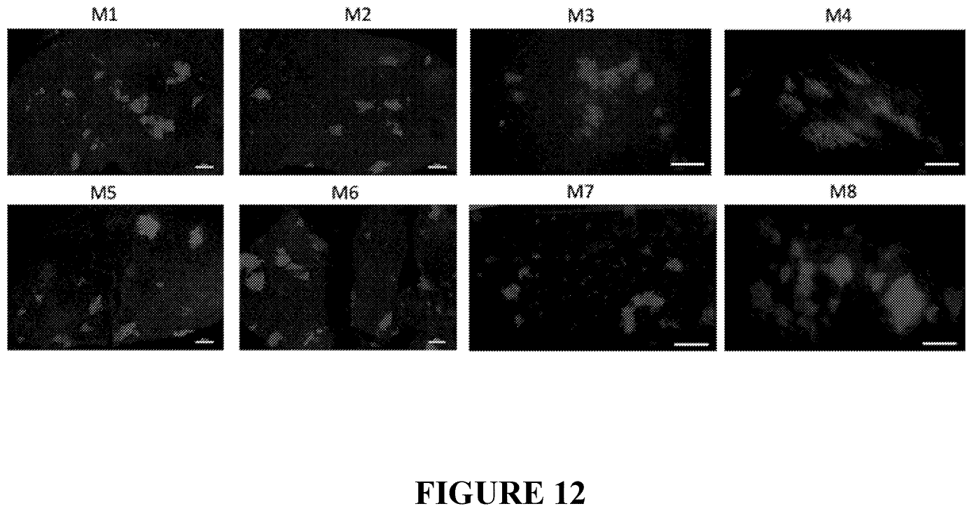

In line with the efficacy of progressive ablation of endogenous lung patch forming cells by BLM, it was found that transplantation of lung cells from TdTomato positive donors into BLM treated mice (FIG. 2A), failed to induce substantial level of donor derived lung patches upon conditioning for 1-3 weeks with BLM, while 4-5 weeks treatment enabled successful chimerism induction with robust donor derived lung patches. This was shown by quantitative analysis of the number of donor-derived patches per 2 mm² lung area in the lungs of recipient mice (FIGS. 2B-2C). In addition, the different levels of chimerism following transplantation after treatment with BLM for different time periods is illustrated in two mice from each group by views of large lung areas (FIG. 2D). Thus, following lung harvest and prior to fixing and freezing, the whole lung tissue was imaged by immunofluorescence microscopy. An additional 8 examples of such large views depicting the variability of chimerism among 20 mice transplanted after 4 weeks of BLM treatment are shown in FIG. 12. These results strongly support the concept of stem cell competition, suggesting that the ablation of endogenous lung forming progenitor cells attained upon fibrosis induction following BLM treatment for 4-5 weeks, allows for effective engraftment and colonization of donor derived lung patches without the need for additional conditioning with TBI or other toxic agents.

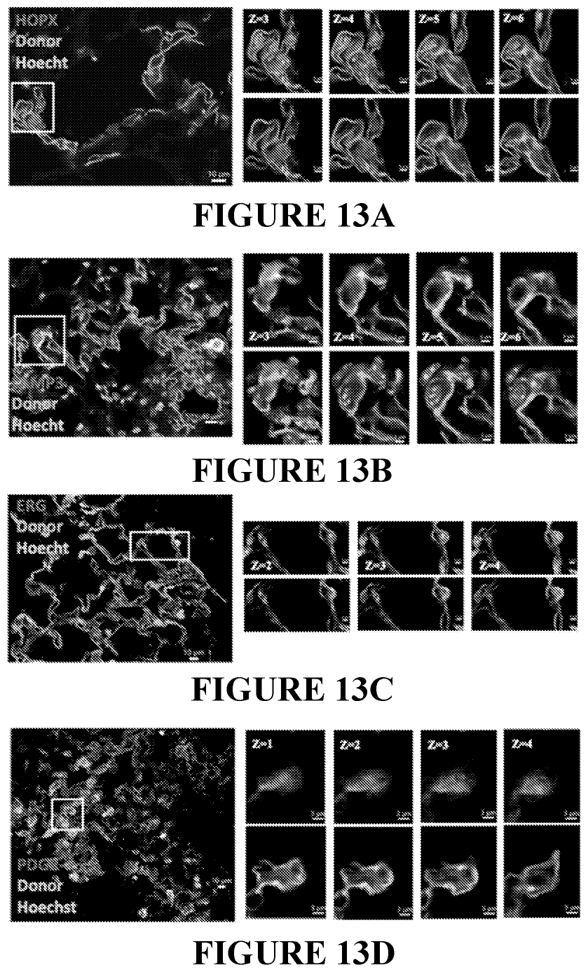

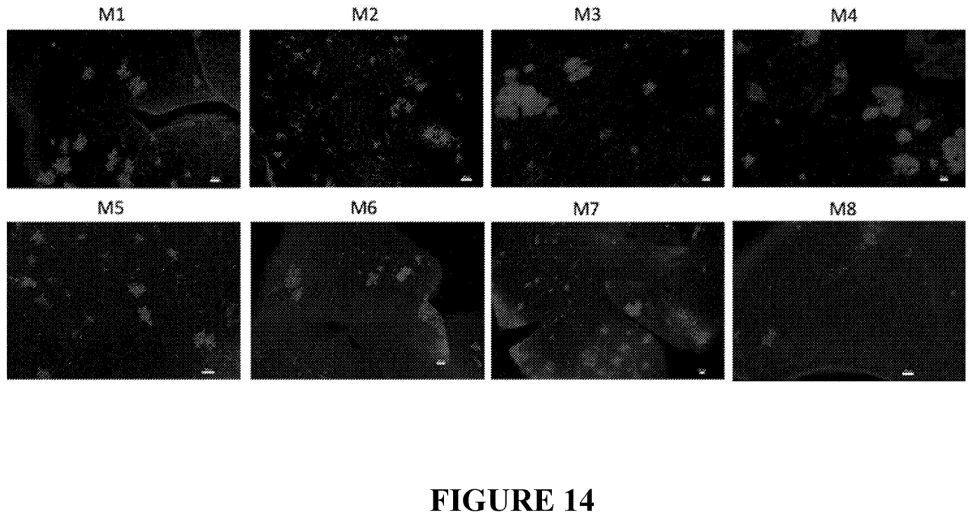

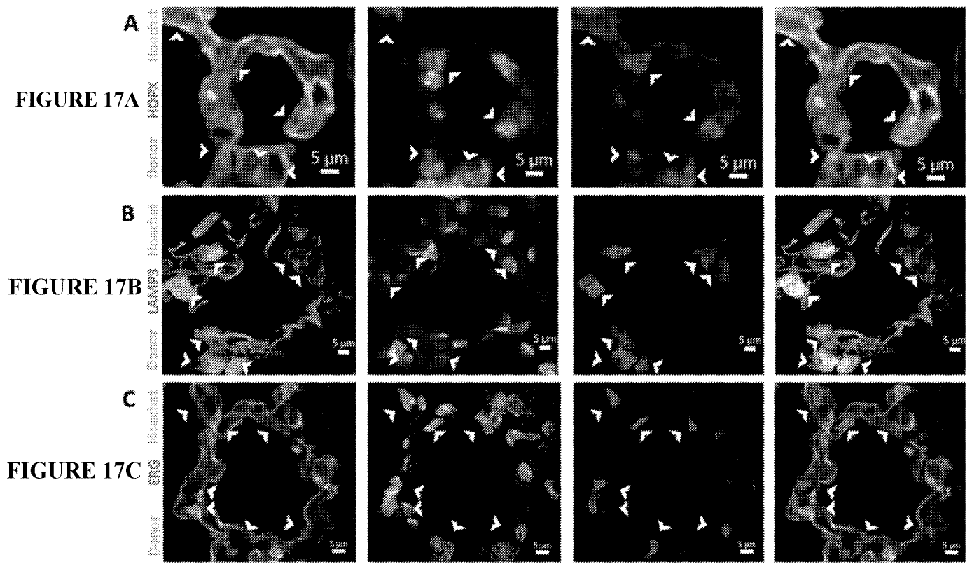

Next, the different cell types comprising the donor derived patches following transplantation into mice treated with BLM for 4 weeks were identified by immunohistology. In these experiments GFP+ donors were used for transplantation and as can be seen in FIGS. 3A-3D, immuno-fluorescence analysis of donor derived patches using double staining revealed that these patches comprised HOPX+ AT1 alveola cells and LAMP3+ AT2 alveolar cells which are of special importance for preventing fibrosis progression in IPF. These patches also included ERG+ endothelial cells and PDGRa+ mesenchymal cells. Further verification of staining along the z axis was performed to rule out potential errors in tracking the boundaries of each donor derived cell (FIGS. 13A-13D). Notably, AT1, AT2 and endothelial cells are incorporated into ells are incorporated into alveoli structures in typical donor-derived patches (FIG. 17). As described above, the robust AT1 and AT2 lung chimerism could be of particular relevance for IPF therapy.

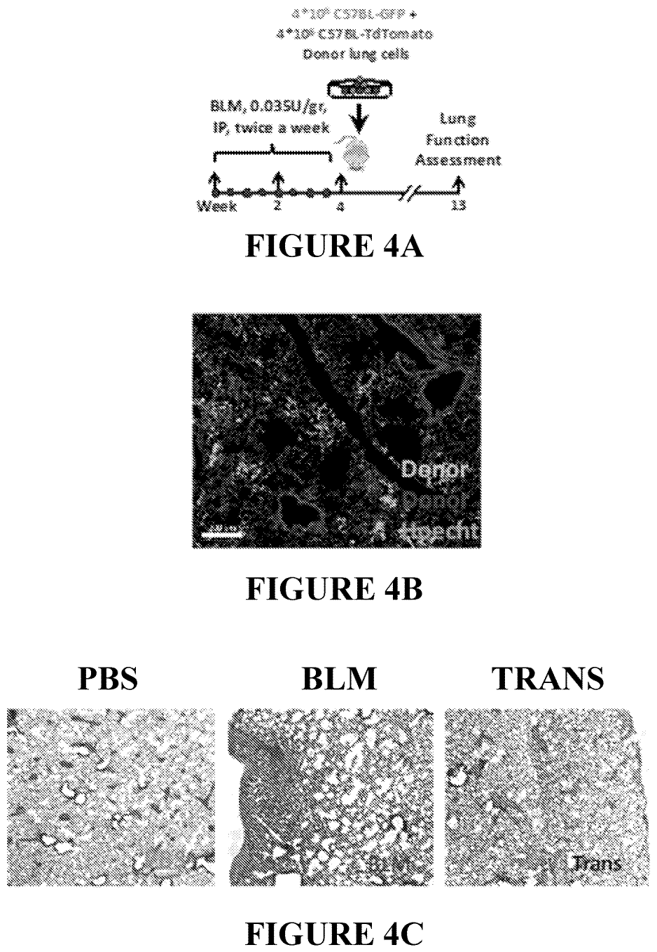

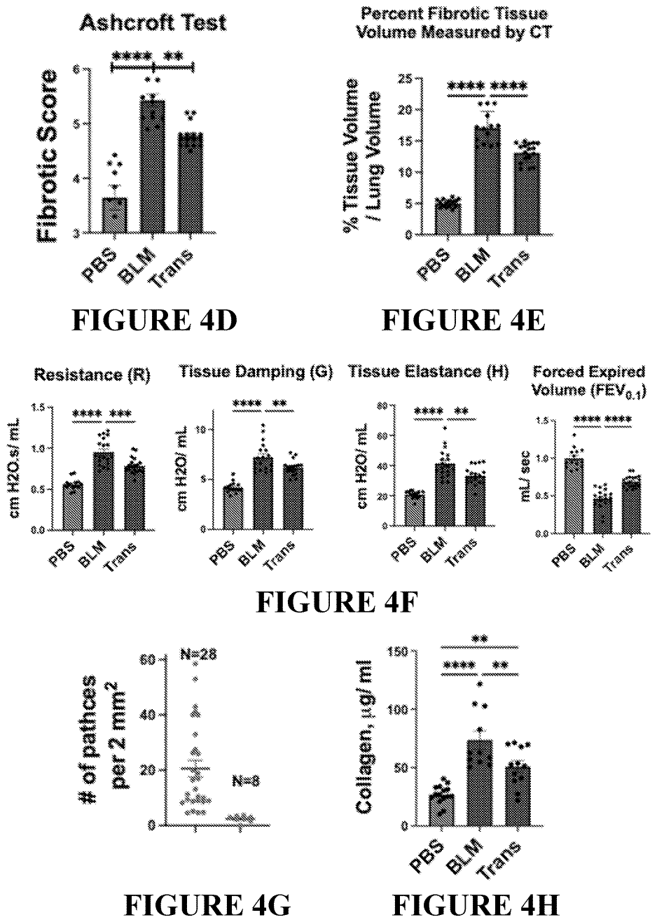

Notably, the marked engraftment of donor derived patch-forming lung forming progenitor cells attained upon transplantation at 1 week after 4 weeks of BLM treatment (FIGS. 4A-4B) was associated with reduced fibrosis compared to BLM treated mice that did not receive a lung cell transplant (FIGS. 4C-4F). Thus, the Ashcroft test measuring tri-chrome staining (FIG. 4D) and the CT analysis (FIG. 4E) revealed remarkably lower levels of fibrosis in the transplanted compared to the non-transplanted group (P<0.002 and P<0001, respectively).

Furthermore, the reduced level of fibrosis was also associated with statistically improved lung function, measured by forced expired volume (P<0002), commonly used to assess function in IPF patients, Resistance (P<0002), Tissue Dumping (P<0.002) and Tissue Elastance (P<0.03) (FIG. 4F).

### Example 3

### Proof of Concept for the Efficacy of Lung Cell Transplantation in the TRF1/SPC Mouse Model

Although the BLM mouse model is used extensively for testing new potential agents for treatment of lung fibrosis, a more relevant model has been described recently by Povedano J. et. al., Cell Rep, 2015; 12(2): 286-99 and Naikawadi et al., JCI Insight, 2015; 1(14): e86704. While the BLM model can be easily used to simulate clinical fibrosis, it is associated with a wide range of toxicities to other tissues and organs including the hematopoietic system and it is not sufficient for studying lung cell damage only. In contrast, the SPC-Cre TRF1^{fl/fl} model, where lung fibrosis occurs spontaneously following tamoxifen treatment, offers a more clinically relevant model of lung fibrosis, and requires extensive breeding, but it can offer a more specific and stable simulation for IPF in which there is clear correlation with shortening of telomers and senescence of AT2 alveolar cells. Thus, in this model deletion of Trf1 in SPC+AT2 cells leads to progression of fibrosis over prolonged period of time, allowing to assess the role of lung stem cell transplantation at late time points following transplantation.

In this model, TRF1 is knocked down specifically in AT2 cells upon treatment with Tamoxifen (TMX), thereby inducing senescence in these cells which in turn leads to progression of fibrosis. Thus, this model simulates more closely the fibrosis found in IPF patients, in whom disease progression was found to be associated with alterations in telomere length.

As shown in FIGS. 5A-5C, chimerism induction after transplantation in the SPC-Cre TRF1^{fl/fl} model, similarly to the BLM model, also depends on progression of fibrosis. Senescence in these cells was shown to lead to progressive lung fibrosis (FIGS. 5E, 5F, and 5H). Thus, this model more closely reproduces the fibrosis found in IPF patients, in whom disease is sometimes associated with defects in telomere maintenance.

As shown in FIGS. 5B, 5C, and 5G, the degree of chimerism induction in the SPC-Cre TRF1fl/fl model, similarly to the BLM model, correlates with progression of fibrosis. Lung cell transplantation at 2 or 4 weeks after completion of TMX course (total 8-10 weeks from the initiation of the experiment) did not lead to notable chimerism, while transplantation 6-8 weeks after completion of TMX treatment (total 12-14 weeks), resulted in marked levels of donor derived patches, without any need for additional conditioning.

The time course of host progenitor ablation was shown by quantitative analysis of the number of donor-derived patches per 2 mm² lung area in the lungs of recipient mice (FIGS. 5E and 5G). Also, the different levels of chimerism following transplantation at different time points following completion of TMX treatment is illustrated per group by low power views of the lung (FIG. 5C). An additional 8 examples of such views representing the variability of chimerism among 16 mice transplanted 7 weeks after completion of TMX treatment are shown in FIG. 14.

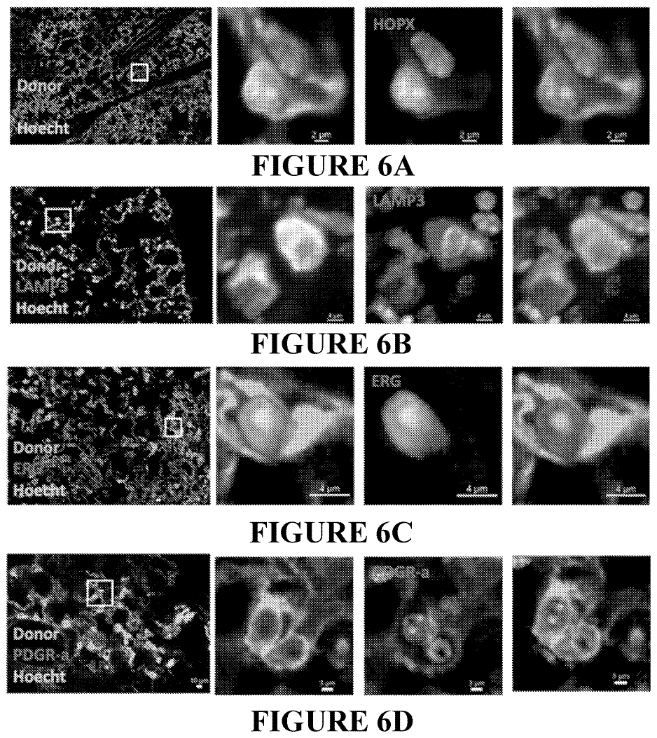

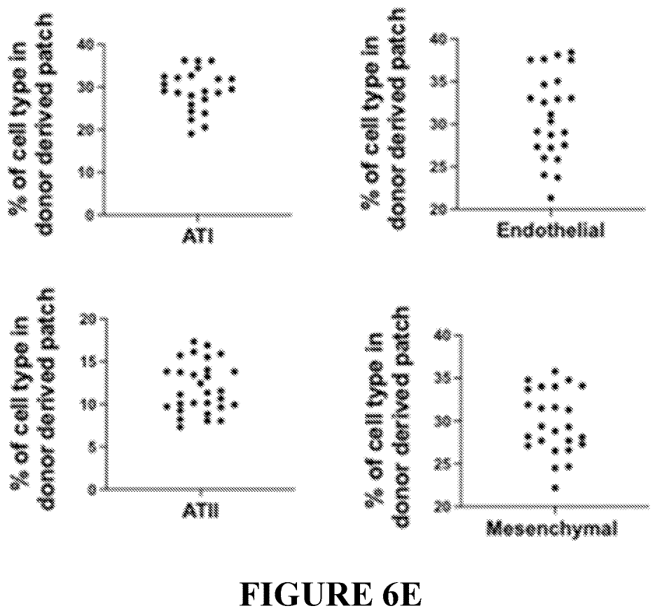

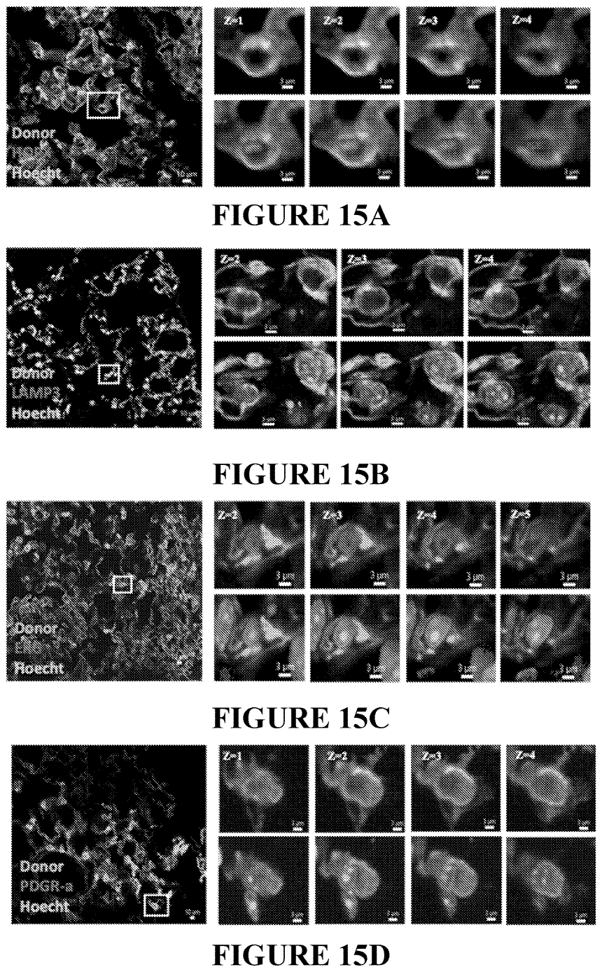

To assess engrafted donor derived patches obtained following transplantation at 14 weeks after initiation of TMX treatment, mice were transplanted with lung cells from C57BL/6- GFP+ donors. As described for the BLM model, quantitative chimerism was assessed at 8 weeks post-transplantation. FIGS. 6A-6D depict typical donor derived patches with epithelial AT1 (HOPX+) and AT2 (LAMP3+) cells, endothelial (ERG+) cells and PDGRa+ mesenchymal cells are clearly observed by immune-histology. Further verification of staining along the z-axis was used to rule out potential errors in tracking the boundaries of each donor derived cell (FIGS. 15A-15D). Quantitative analysis showing the distribution of these cell types in more than 40 patches from 3-5 chimeric mice is shown in FIG. 6E.

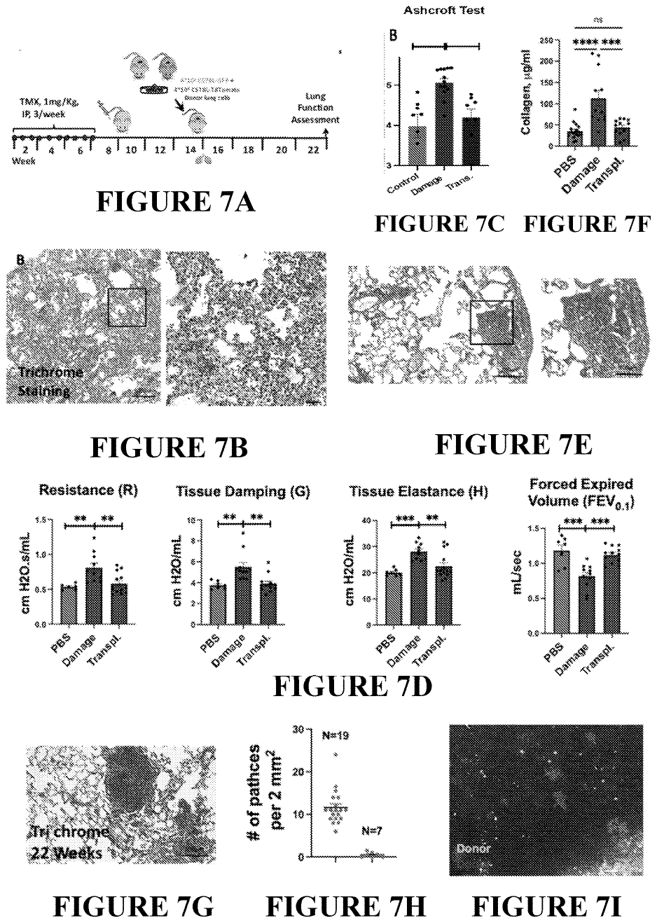

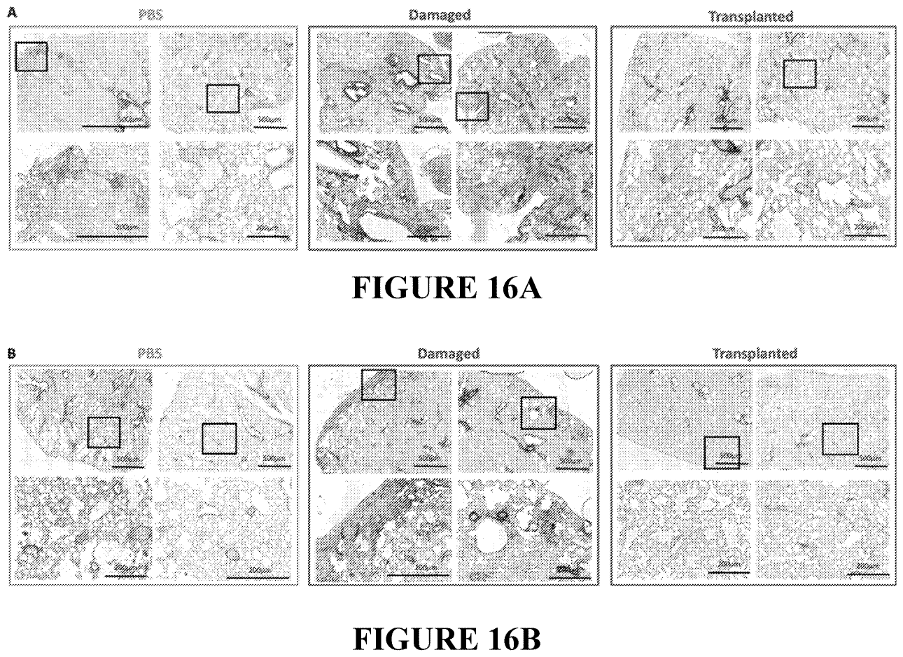

Finally, transplanted SPC-Cre TRF1^{fl//fl} mice as outlined in FIG. 7A. exhibited successful engraftment of donor patch forming cells associated with marked prevention of fibrosis progression as indicated by the Ashcroft test (FIGS. 7B-7C), lung functional tests (FIG. 7D), hyaluronic acid staining (FIGS. 16A-16B), and collagen levels (FIG. 7F).

Taken together, based on two distinct models of fibrosis it was concluded that a certain level of fibrosis induction is required to ablate the endogenous stem cell pool and to reduce stem cell competition with donor derived lung stem cells.

Thus, in both the BLM and the TRF1/SPC models it was found that a certain minimal level of fibrosis is required to allow effective engraftment of donor derived patch forming lung forming progenitor cells without any need for additional conditioning. Furthermore, it was demonstrated in the BLM model that this minimal level is associated with marked elimination of host patch forming cells in line with our hypothesis that overcoming stem cell competition is a prerequisite for effective engraftment. Effective chimerism induction was demonstrated to attenuate fibrosis and provided functional benefits in both models. These results offer a proof of concept for the clinical use of lung stem cell transplantation in patients with lung fibrosis.

### Example 4

### Pre-Conditioning Allows Engraftment of Embryonic Lung Cells

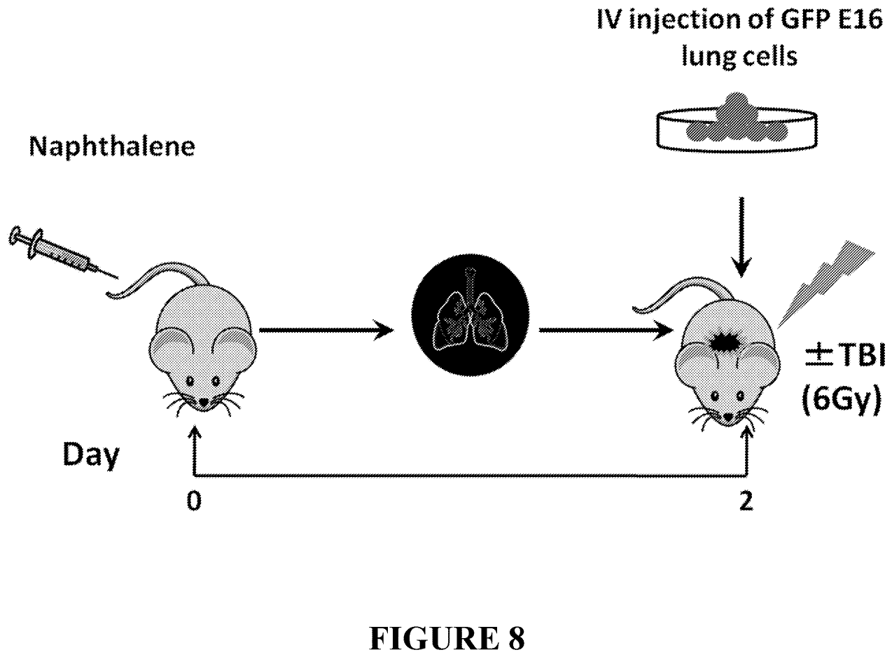

In contrast to the above mentioned lung fibrosis mouse models it was found in the naphthalene (NA) lung injury model pre-conditioning is required for induction of lung chimerism. (FIG. 8). Thus, mice were treated with NA alone or with NA plus 6 Gy total body irradiation (TBI) 48 hours after naphthalene treatment. The mice were infused with 1×10⁶ lung cells from E16 GFP^{+ donors and were monitored for engraftment and development of donor-derived cells using immunohistological staining, morphometric analysis, and two-photon microscopy.}

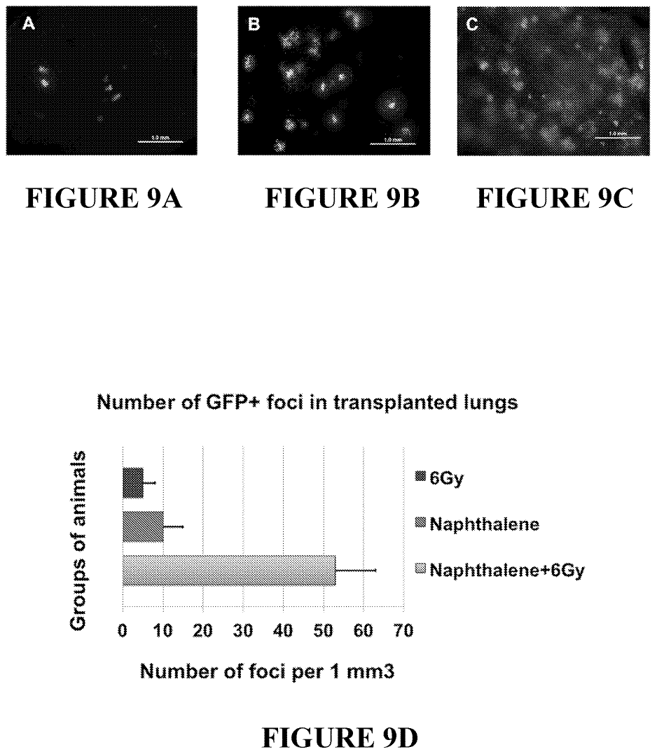

FIGS. 9A-9C provide representative fluorescence microscopy images of mouse lungs following transplantation into TBI treated, NA treated or NA+TBI treated. FIG. 9A shows a fluorescence microscopy image of lungs of C57BL/6 adult mouse conditioned with 6 GY TBI alone and transplanted with GFP⁺ embryonic precursor lung cells. FIG. 9B shows a fluorescence microscopy image of lungs of C57BL/6 adult mouse treated with NA alone and transplanted with GFP⁺ embryonic precursor lung cells. FIG. 9C shows a fluorescence microscopy image of lungs of C57BL/6 adult mouse treated with NA, 48 hour later conditioned with 6 GY TBI and transplanted with GFP⁺ embryonic precursor lung cells. FIG. 9D shows results from quantitative morphometric analysis of GFP⁺ patches of engrafted cells per mm³ lung tissue after 6 Gy radiotherapy, naphthalene, and 6 Gy radiotherapy plus napthalene conditioning regimens. GFP⁺ patches, indicating engraftment of donor-derived cells in the recipient lungs, were markedly enhanced after transplantation in mice pre-conditioned with naphthalene and TBI compared to those conditioned with TBI or naphthalene only, suggesting that the combined preconditioning regimen enhanced donor cell engraftment.

### Example 5

### Adaptation of a BLM Mouse Model for the Evaluation of Stem Cell Therapy

Lung fibrosis induced in several BLM mouse models was shown to be reversible. It was therefore critical to develop a more enduring model to allow for the assessment of lung stem cell transplantation efficacy, which requires a period of about 6-8 weeks post transplantation to stop deterioration of lung function. Thus, the present study adopted and optimized the protocol described by Headley et al. (Exp Physiol, 2018; 103(12): 1692-1703). This protocol uses a low dose BLM administered systematically over a period of four weeks in order to induce more stable lung fibrosis and to enable the benefits of the transplanted cells to be assessed at relatively late time points following cell transfer.

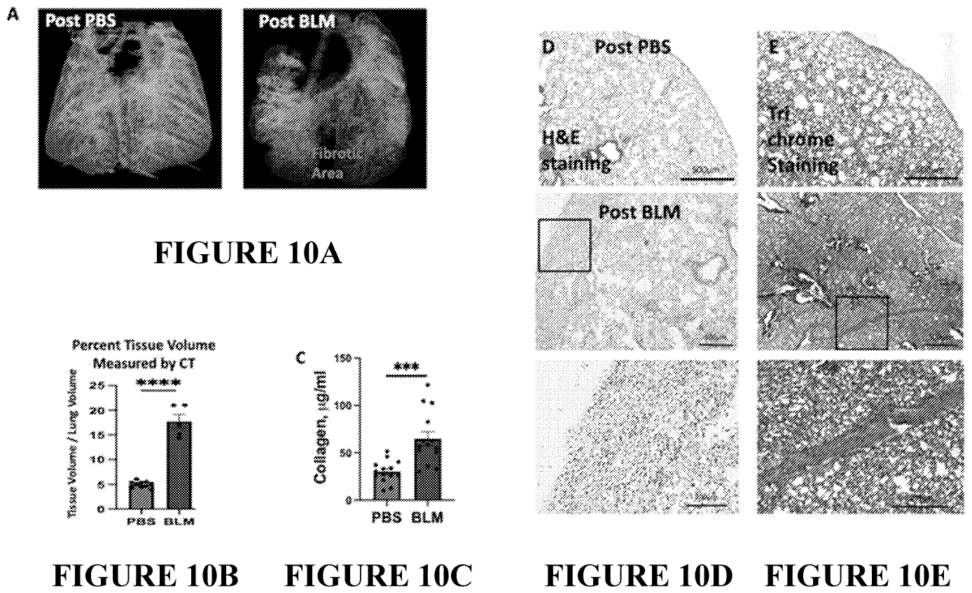

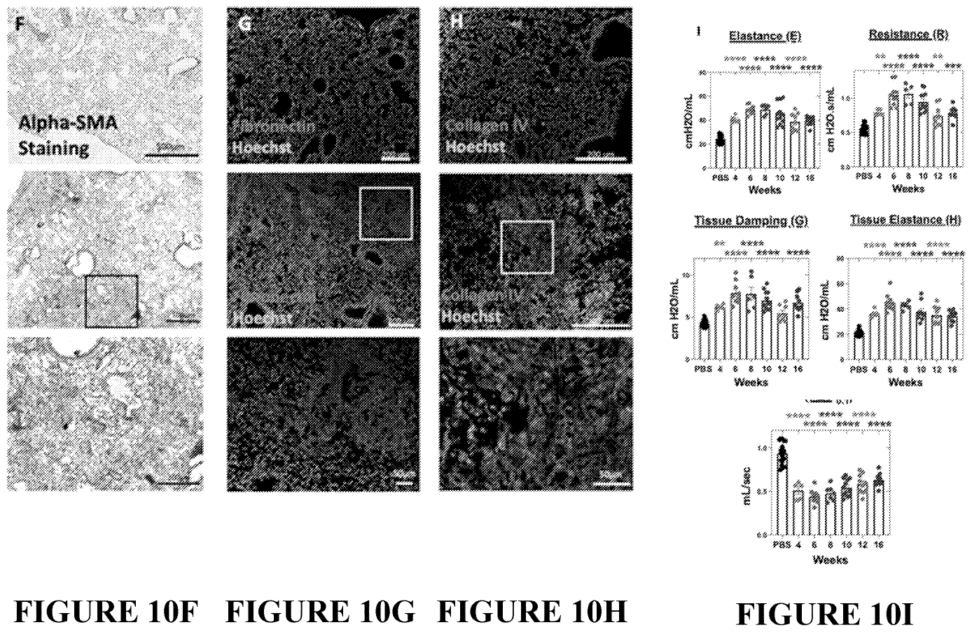

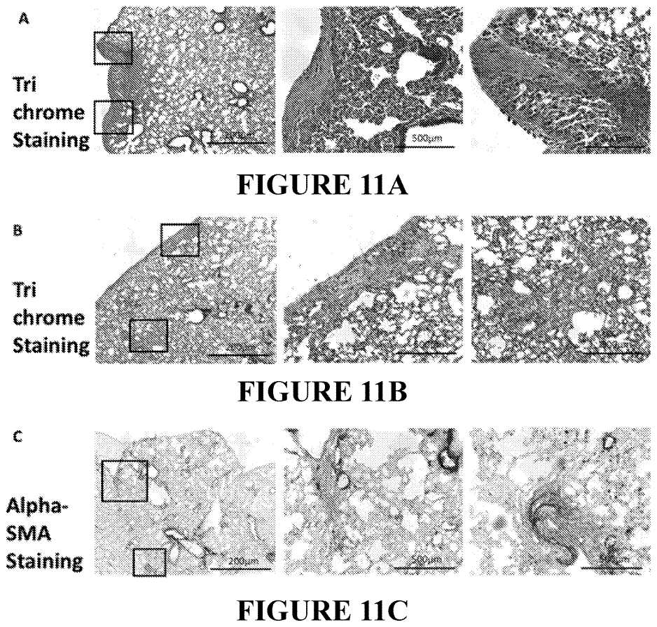

As shown in FIG. 10, i.p. administration of BLM for 4 weeks (total of 8 doses each of 0.035 U/b) led to marked lung fibrosis, detected by CT (FIG. 10A-10B) and by the quantitative hydroxyproline assay for collagen levels at 8 weeks after completion of BLM treatment (FIG. 10C). Furthermore, substantial fibrosis was indicated by H&E (FIG. 10D), Trichrome (FIGS. 10E and 11A-11B), Alpha Sma (FIGS. 10F and 11C), Fibronectin (FIG. 10G) Collagen IV staining (FIG. 10H), and hyaluronic acid (HA, FIG. 16A) staining. Notably, analysis of lung function parameters measured by Flexivent at different time points after completion of BLM treatment revealed maximal loss of function at 6-8 weeks following completion of BLM administration (FIG. 10I). These results demonstrate that the BLM-induced lung damage is stable over a prolonged period, allowing the therapeutic effect of our suggested lung transplantation modality to be evaluated at 2 months post-transplantation if performed after 4 weeks of BLM administration Although the invention has been described with reference to the above examples, it will be understood that modifications and variations are encompassed within the spirit and scope of the invention. Accordingly, the invention is limited only by the following claims.

1. A method of treating pulmonary fibrosis in a subject comprising:
administering to a subject having at least about 15% lung fibrosis a therapeutically effective amount of lung forming progenitor cells, thereby treating pulmonary fibrosis in the subject.

2. The method of claim 1, wherein prior to administering, a level of fibrosis in the lung of the subject is measured by comparing a fibrosis volume in the lung to a total lung volume, wherein measuring comprises subjecting the subject to a computed tomography (CT) scan and/or assessing the level of fibrosis by a lung biopsy sample.

3-4. (canceled)

5. The method of claim 1, wherein treatment is administered in the absence of a pre-conditioning treatment prior to the administration of the cells.

6. The method of claim 1, further comprising dissociating a pulmonary tissue to obtain lung forming progenitor cells.

7-9. (canceled)

10. The method of claim 1, wherein the cells are autologous cells or allogeneic cells.

11-12. (canceled)

13. The method of claim 6, further comprising enriching the cells for those expressing an endothelial marker, wherein the endothelial marker is CD31, CD144, or ERG; and/or an epithelial marker, wherein the epithelial marker is selected from CD326, CD324, CD245, aquaporin-5, podoplanin, or advanced glycosylation end-product specific receptor; depleting the cells of cells expressing CD45, or a combination thereof.

14-17. (canceled)

18. The method of claim 13, further comprising enriching the cells for those that are CD326+CD31+, CD324+CD31+, CD326+CD144+, and/or CD324+CD144+.

19. The method of claim 13, further comprising depleting the cells of T cells, depleting the cells of B cells, or a combination thereof.

20. The method of claim 13, wherein enriching the cells or depleting the cells comprises contacting the cells with an agent that binds to the epithelial marker, the endothelial marker or CD45.

21-22. (canceled)

23. The method of claim 1, further comprising determining expression of an epithelial marker, an endothelial marker, or a combination thereof in the cells;
wherein the epithelial marker is selected from CD326, CD324, CD245, aquaporin-5, podoplanin, or advanced glycosylation end-product specific receptor; and wherein the endothelial marker is CD31, CD144, or ERG.

24-27. (canceled)

28. The method of claim 23, wherein a predetermined proportion of the cells express the epithelial marker and the endothelial marker, wherein the predetermined proportion is least about 0.1%, at least about 1%, at least about 2%, at least about 5%, at least about 10%, at least about 20%, at least about 30%, at least about 40%, at least about 50%, at least about 60%, at least about 70%, at least about 80%, at least about 90%, or at least about 95% of the cells.

29-30. (canceled)

31. The method of claim 23, wherein the cells are expanded for at least about 12 hours, at least about 24 hours, at least about 36 hours, at least about 48 hours, at least about 72 hours, at least about 120 hours, at least about 180 hours, at least about 240 hours, at least about 300 hours, at least about 600 hours, or least about 1200 hours prior to the determining.

32. The method of claim 23, wherein the cells are determined to not be suitable for administration to the subject when a proportion of the cells which do not express the epithelial marker and the endothelial marker exceeds a predetermined threshold;
wherein the predetermined threshold is at least about 0.1%, at least about 1%, at least about 2%, at least about 5%, at least about 10%, at least about 20%, at least about 30%, at least about 40%, at least about 50%, at least about 60%, at least about 70%, at least about 80%, at least about 90%, or at least about 95% of the cells.

33. (canceled)

34. The method of claim 1, wherein the subject has idiopathic pulmonary fibrosis or chronic obstructive pulmonary disease.

35. (canceled)

36. A method of improving lung function in a subject in need thereof comprising:
administering to a subject having at least about 15% lung fibrosis a therapeutically effective amount of lung forming progenitor cells, thereby improving lung function in the subject.

37. (canceled)

38. The method of claim 36, wherein improving lung function comprises reducing fibrosis and/or preventing or slowing down fibrosis progression; and/or improving respiratory system Newtonian resistance, lung tissue compliance, lung tissue elastance, tissue dampening and/or forced expired volume.

39-42. (canceled)

43. A method of identifying a subject with pulmonary fibrosis as a candidate for treatment with lung forming progenitor cells comprising:
a) measuring the level of fibrosis in the lungs of the subject; and
i) classifying a subject with a level of fibrosis of at least about 15% or greater as a likely responder to cell transplantation, thereby identifying the subject as suitable for cell transplantation, or
ii) classifying a subject with a level of fibrosis of about 0 to less than 15% as a likely non-responder to lung cell transplantation, thereby identifying the subject as unsuitable for cell transplantation,
thereby identifying a subject with pulmonary fibrosis as a candidate for treatment with lung forming progenitor cells.

44. (canceled)

45. The method of claim 43, further comprising administering a cell suspension comprising isolated lung forming progenitor cells to the subject identified as a likely responder; wherein administering the cell suspension comprises increasing a number of donor-derived patch forming cells and/or decreasing a number of host-derived patch-forming cells.

46. (canceled)

47. The method of claim 43, wherein the donor-derived patch forming cells express both endothelial and epithelial markers; wherein the endothelial marker is CD31, CD144, or ERG and the epithelial marker is CD326, CD324, CD245, aquaporin-5, podoplanin, or advanced glycosylation end-product specific receptor.

48-51. (canceled)

52. The method of claim 43, further comprising measuring a second level of fibrosis in the lungs of the subject at a second time point and (i) maintaining the subject as an unlikely responder if the second level of fibrosis is of about 0 to less than 15%; or (ii) reclassifying the subject as a likely responder if the second level of fibrosis is of at least about 15% or greater.

53. The method of claim 43, wherein a level of fibrosis of at least about 15% or greater is indicative of a reduction in a number of endogenous lung stem cells sufficient to ensure engraftment of the lung forming progenitor cells, and wherein a level of fibrosis of about 1 to less than 15% is indicative of an absence of a sufficient reduction in a number of endogenous lung stem cells to allow engraftment of the lung forming progenitor cells.

54. (canceled)

55. The method of claim 1, further comprising administering the subject an immunosuppressive therapy, wherein the immunosuppressive therapy comprises tacrolimus, everolimus, sirolimus, rapamycin, cyclosporine A, an anti-lymphocyte globulin antibody, an anti-thymocyte globulin (ATG) antibody, an anti-CD3 antibody, a steroid, azathioprine, an anti-IL-2Ra receptor antibody, mycophenolic acid, or an anti-CD20 antibody.

56. (canceled)
